SEQUENCE LISTING (42 sequences)

SEQ ID NO: 1 - protein, 16 aa, Artificial
  peptide derived from HIV-1 reverse transcriptase
GTKALTEVIPLTEEAE

SEQ ID NO: 2 - protein, 16 aa, Artificial
  peptide derived from HIV-1 reverse transcriptase
ATKALTEVIPLTEEAE

SEQ ID NO: 3 - protein, 16 aa, Artificial
  peptide derived from HIV-1 reverse transcriptase
GAKALTEVIPLTEEAE

SEQ ID NO: 4 - protein, 16 aa, Artificial
  peptide derived from HIV-1 reverse transcriptase
GTAALTEVIPLTEEAE

SEQ ID NO: 5 - protein, 16 aa, Artificial
  peptide derived from HIV-1 reverse transcriptase
GTKGLTEVIPLTEEAE

SEQ ID NO: 6 - protein, 16 aa, Artificial
  peptide derived from HIV-1 reverse transcriptase
GTKAATEVIPLTEEAE

SEQ ID NO: 7 - protein, 16 aa, Artificial
  peptide derived from HIV-1 reverse transcriptase
GTKALAEVIPLTEEAE

SEQ ID NO: 8 - protein, 16 aa, Artificial
  peptide derived from HIV-1 reverse transcriptase
GTKALTAVIPLTEEAE

SEQ ID NO: 9 - protein, 16 aa, Artificial
  peptide derived from HIV-1 reverse transcriptase
GTKALTEAIPLTEEAE

SEQ ID NO: 10 - protein, 16 aa, Artificial
  peptide derived from HIV-1 reverse transcriptase
GTKALTEVAPLTEEAE

SEQ ID NO: 11 - protein, 16 aa, Artificial
  peptide derived from HIV-1 reverse transcriptase
GTKALTEVIALTEEAE

SEQ ID NO: 12 - protein, 16 aa, Artificial
  peptide derived from HIV-1 reverse transcriptase
GTKALTEVIPATEEAE

SEQ ID NO: 13 - protein, 16 aa, Artificial
  peptide derived from HIV-1 reverse transcriptase
GTKALTEVIPLAEEAE

SEQ ID NO: 14 - protein, 16 aa, Artificial
  peptide derived from HIV-1 reverse transcriptase
GTKALTEVIPLTAEAE

SEQ ID NO: 15 - protein, 16 aa, Artificial
  peptide derived from HIV-1 reverse transcriptase
GTKALTEVIPLTEAAE

SEQ ID NO: 16 - protein, 16 aa, Artificial
  Srambled peptide
GAKTETLVIPETELEA

SEQ ID NO: 17 - protein, 16 aa, Artificial
  peptide derived from HIV-1 reverse transcriptase
GTKWLTEWIPLTAEAE

SEQ ID NO: 18 - protein, 16 aa, Artificial
  peptide derived from HIV-1 reverse transcriptase
GTKWLTEWIPLTAEAE

SEQ ID NO: 19 - protein, 11 aa, Artificial
  peptide derived from HIV-1 reverse transcriptase
GTKWLTEVWPL

SEQ ID NO: 20 - protein, 11 aa, Artificial
  peptide derived from HIV-1 reverse transcriptase
GTKAWTEVWPL

SEQ ID NO: 21 - protein, 12 aa, Artificial
  peptide derived from HIV-1 reverse transcriptase
GTKALTEVIPLT

SEQ ID NO: 22 - protein, 12 aa, Artificial
  peptide derived from HIV-1 reverse transcriptase
GTKAATEVIPLT

SEQ ID NO: 23 - protein, 11 aa, Artificial
  peptide derived from HIV-1 reverse transcriptase
GTKWLTEWIPL

SEQ ID NO: 24 - protein, 14 aa, Artificial
  peptide derived from HIV-1 reverse transcriptase
KWLTEWIPLTAEAE

SEQ ID NO: 25 - protein, 14 aa, Artificial
  peptide derived from HIV-1 reverse transcriptase
GTKWLTEWIPLTAE

SEQ ID NO: 26 - protein, 16 aa, Artificial
  peptide derived from HIV-1 reverse transcriptase
GTKWATEWAPLTAEAE

SEQ ID NO: 27 - protein, 12 aa, Artificial
  peptide derived from HIV-1 reverse transcriptase
KWLTEWIPLTAE

SEQ ID NO: 28 - protein, 17 aa, Artificial
  peptide derived from HIV-1 reverse transcriptase
RGTKALTEVIPLTEEAE

SEQ ID NO: 29 - protein, 21 aa, Artificial
  peptide vector
KETWWETWWTEWSQPKKKRKV

SEQ ID NO: 30 - DNA, 19 bp, Artificial
  primer strand
tccctgttcgggcgccact

SEQ ID NO: 31 - DNA, 36 bp, Artificial
  template strand
tgtggaaaatctcatgcagtggcgcccgaacaggga

SEQ ID NO: 32 - protein, 10 aa, Artificial
  Pep-7 ; described in Morris et al., 1999, JBC, 274, 24941-24946
KETWETWWTE

SEQ ID NO: 33 - protein, 15 aa, Artificial
  peptide vector
KWFETWFTEWPKKRK

SEQ ID NO: 34 - protein, 16 aa, Artificial
  peptide derived from HIV-1 reverse transcriptase
GTKWLTEWIPLTAEAE

SEQ ID NO: 35 - protein, 18 aa, Artificial
  Cyclized Paw peptide
CGTKWLTEWIPLTAEAEC

SEQ ID NO: 36 - protein, 17 aa, Artificial
  Cyclized Paw peptide
GCKWLTEWIPLTAEAEC

SEQ ID NO: 37 - protein, 13 aa, Artificial
  Cyclized P16 peptide
CGTKWLTEVWPLC

SEQ ID NO: 38 - protein, 12 aa, Artificial
  Cyclized P16 peptide
GCKWLTEVWPLC

SEQ ID NO: 39 - protein, 16 aa, Artificial
  Cyclized P27 peptide
CGTKWLTEWIPLTAEC

SEQ ID NO: 40 - protein, 15 aa, Artificial
  Cyclized P27 peptide
GCKWLTEWIPLTAEC

SEQ ID NO: 41 - protein, 21 aa, Artificial
  CADY-2c; described in International Application WO 2007/069090
GLWRALWRALWRSLWKKKRKV

SEQ ID NO: 42 - protein, 20 aa, Artificial
  Peptide vector; described in International Application WO 2007/069090
GLWRALWRLLRSLWRLLWKA

(19) United States
(12) Patent Application Publication
(10) Pub. No.: US 2011/0294728 A1
(43) Pub. Date: Dec. 1, 2011

(54) Antiviral Polypeptides

(76) Inventors: Gilles Divita, Mauguio (FR); Audrey Agopian, Montpellier (FR); Edwige Gros, Montpellier (FR)

(21) Appl. No.: 13/121,057

(22) PCT Filed: Sep. 24, 2009
(86) PCT No.: PCT/IB09/07482

§ 371 Date: Aug. 11, 2011

(30) Foreign Application Priority Data
Sep. 26, 2008 (EP) 08290908.6

(51) Int. Cl. A61K 38/10; C07K 7/06; C07K 14/16; A61P 31/18; C07H 21/00; C12N 15/63; C12N 1/21; C12N 1/19; C07K 7/08; C12N 9/99

(52) U.S. Cl. 514/3.8; 530/326; 530/327; 530/324; 435/184; 536/23.72; 435/320.1; 435/252.3; 435/255.1

(57) ABSTRACT

The invention concerns polypeptides derived from the HIV-1 reverse transcriptase which are capable of inhibiting said polymerase and optionally also capable of inhibiting the HIV-1 integrase 3′ processing activity, and their therapeutic applications.

The invention relates to polypeptides derived from the HIV-1 reverse transcriptase for use as antiviral compounds.

Human immunodeficiency virus type I (HIV-1) is the primary cause of acquired immunodeficiency syndrome (AIDS), a slow progressive and degenerative disease of the human immune system. Despite recent therapeutic developments and the introduction of Highly Active Antiretroviral Therapy (HAART), the rapid emergence of drug-resistant viruses against all approved drugs together with inaccessible latent virus reservoirs and side effects of currently used compounds have limited the efficacy of existing anti-HIV-1 therapeutics (Simon et al., 2006). Therefore, there is still an urgent need for new and safer drugs, active against resistant viral strains or directed towards novel targets in the replicative cycle, which will be useful for multiple drug combination.

HIV-1 reverse transcriptase (RT) plays an essential multifunctional role in the replication of the virus, by catalysing the synthesis of double-stranded DNA from the single-strand retroviral RNA genome (di Marzo Veronese et al., 1986; Lightfoote et al., 1986). The majority of the chemotherapeutic agents used in AIDS treatments target the polymerase activity of HIV-1 reverse transcriptase, such as nucleoside reverse transcriptase inhibitors (NRTIs) or non-nucleoside reverse transcriptase inhibitors (NNRTIs) (De Clercq, 2007). The biologically active form of reverse transcriptase is an asymmetric heterodimer that consists of two subunits, p66 and p51 derived from p66 by proteolytic cleavage of the C-terminal RNAse H domain (di Marzo Veronese et al., 1986; Lightfoote et al., 1986; Restle et al., 1990).

The polymerase domain of both p66 and p51 subunit can be subdivided into four common subdomains: fingers, palm, thumb and connection (Kohlstaedt et al., 1992; Jacobo-Molina et al., 1993; Huang et al., 1998; Rodgers et al., 1995; Hsiou et al., 1996). Determination of the three-dimensional structures of reverse transcriptases has revealed that although the folding of individual subdomains is similar in p66 and p51, their spatial arrangement differs markedly (Wang et al., 1994). The p66 subunit contains both polymerase and RNase H active sites. The p66-polymerase domain folds into an “open”, extended structure, forming a large active site cleft with the three catalytic residues (Asp¹¹⁰, Asp¹⁸⁵, Asp¹⁸⁶) within the palm subdomain exposed in the nucleic acid binding site. The primer grip is responsible for the appropriate placement of the primer terminus at the polymerase active site and is involved in translocation of the primer-template (p/t) following nucleotide incorporation (Ghosh et al., 1996; Wohrl et al., 1997 and Patel et al., 1995). In contrast, p51 predominantly plays a structural role in the reverse transcriptase heterodimer, by stabilizing the dimer interface thereby favouring loading of the p66 onto the primer-template and maintaining the appropriate enzyme conformation during initiation of reverse transcription (Huang et al., 1992).

In order to propose new classes of HIV inhibitors, extensive efforts have been made in the design of molecules that target protein/protein interfaces required for viral entry, replication and maturation (Divita et al., 1994, 1995a; Camarasa et al., 2006 and Sticht et al., 2005). The inventors (Divita et al., 1995b and Morris et al., 1999a and 1999b) and others (Restle et al., 1990 and Tachedjian et al., 2001) have proposed that the heterodimeric organization of reverse transcriptase constitutes an interesting target for the design of new inhibitors. The formation of the active heterodimeric HIV-1 reverse transcriptase occurs in a two step process. First a rapid association of the two subunits (dimerization-step) via their connection sub-domains thereby yielding an inactive intermediate-reverse transcriptase, followed by a slow conformational change of this intermediate (maturation-step), generating the biologically active form of this enzyme. The maturation step involves contacts between the thumb of p51 and the RNAse H of p66 as well as between the fingers of p51 with the palm of p66 (Divita et al., 1995b; Cabodevilla et al., 2001; Morris et al., 1999a and Depollier et al., 2005). NNRTIs have been reported to interfere with reverse transcriptase dimerization and to modulate the overall stability of the heterodimeric reverse transcriptase depending on their binding site on reverse transcriptase (Tachedjian et al., 2001; Venezia et al., 2006; Sluis-Cremer, and Tachedjian, 2002; Sluis-Cremer et al., 2002 and Tachedjian and Goff, 2003). NNRTIs including Efavirenz and Nevirapine have been shown to promote HIV-1 reverse transcriptase maturation at the level of the Gag-Pol protein and to affect viral protease (PR) activation, resulting in the suppression of viral release from infected cells (Tachedjian et al., 2005 and Figueiredo et al., 2006). Conversely, NNRTIs such as TSAO and BBNH derivatives act as destabilizers of reverse transcriptase subunit interaction (Sluis-Cremer et al., 2002).

The inventors have demonstrated that preventing or controlling reverse transcriptase dimerization constitutes an alternative strategy to block WV proliferation and has a major impact on the viral cycle (Morris et al., 1999b). In protein-protein interactions the binding energy is not evenly distributed across the dimer interface but involves specific residues “hot spots” that stabilize protein complexes. The inventors have also shown that the use of small peptides targeting “hot spot” residues required for reverse transcriptase-dimerization constitutes a new strategy to inhibit HIV-1 reverse transcriptase (Divita et al., 1994 and 1995a) and have described a decapeptide “p7” (or “Pep-7”, referred herein as SEQ ID NO: 32) mimicking p66/p51 interface that prevents reverse transcriptase-dimerization by destabilizing reverse transcriptase subunit-interactions and that blocks viral replication (Depollier et al., 2005 and Morris et al., 1999b; International Application WO 02/15661).

The thumb domain plays an important role in the catalysis and integrity of the dimeric form of reverse transcriptase, thereby constituting a potential target for the design of novel antiviral compounds (Jacobo-Molina et al., 1993; Huang et al., 1998 and Morris et al., 1999a). The p66-thumb domain is involved in primer-template binding and polymerase activity of reverse transcriptase (Jacobo-Molina et al., 1993; Huang et al., 1998 and Wohrl et al., 1997). The p51-thumb domain is required for the conformational changes associated with reverse transcriptase dimer-maturation (Morris et al., 1999a). The Inventors have designed a peptide, Pep-A (SEQ ID NO: 28), derived from a structural motif located between residues 284 and 300, corresponding to the end of helix al, the loop connecting helices αI and αJ and a part of helix αJ. This peptide is a potent inhibitor of reverse transcriptase interfering with the conformational change associated with full activation of the enzyme. However, although it significantly blocks reverse transcriptase maturation in vitro, it lacks antiviral activity (Morris et al., 1999a).

A new generation of HIV inhibitors targeting the third viral enzyme HIV-1 integrase (IN) has been introduced into clinics (Grinsztejn et al., 2007). Integrase is a 32 kDA viral protein that is biologically active under a multimeric form (Cherepanov et al., 2003 and Guiot et al., 2006). Its oligomeric status is dependent on the reaction catalyzed. A dimeric form is required at each end of the viral DNA end for 3′ processing while the tetramer is responsible for the concerted integration (Guiot et al., 2006). Integrase has been reported to be involved in numerous protein/protein interactions with both viral and cellular proteins within the PIC. Integrase plays an essential role in the life cycle of HIV, by catalyzing the insertion of the viral DNA into the host cell chromosome, throughout a multi-steps process taking place during or immediately after reverse transcription of the viral genomic RNA (Craigie, 2001 and Semenova et al., 2008). Integrase binds the viral DNA, thereby forming a nucleoprotein complex, which constitutes the main component of the preintegration complex (PIC). Although the composition of the PIC still remains to be clarified, it contains viral components such as Reverse Transcriptase and Vpr as well as cellular components like BAF, LEDGF-p75, INI1, HMGA1 that stabilize IN/DNA complex, favor its nuclear import and improve integrase mediated viral DNA insertion (Depienne et al., 2000; Farnet et al., 1997; Piller et al., 2003; Popov et al., 1998; Semenova et al., 2008 and Van Maele and Debyser, 2005). Within the PIC, integrase performs 3′-processing in the cytoplasm, which consists in the cleavage of two terminal nucleotides from both 3′-ends of viral DNA. Then, in the nucleus of infected cell, integrase mediates a strand transfer reaction that inserts viral DNA into the cell DNA, resulting in a full-site integration.

The integrity of complexes involving integrase is a requirement for viral replication (Piller et al., 2003 and Semenova et al., 2008). Therefore, altering integrase structural organization and its interactions with partners offer potent targets for the design of new inhibitors. To this aim, extensive efforts have been made to design molecules that target structural organization of the enzyme (Semenova et al., 2008). Inhibitors including monoclonal antibodies, oligonucleotide conjugates and peptides have been proposed to constrain the structure of integrase or to disrupt or prevent oligomeric integrase structure or its complex with DNA (Semenova et al., 2008 and Pommier et al., 2005).

The most promising integrase inhibitors reported so far inhibit the strand transfer reaction, by interacting directly with the active site of the preassembled IN/viral DNA complex (Pommier et al., 2005). Two strand transfer inhibitor compounds, Raltegravir (MK-0518) and Elvitegravir (GS-9137), are currently in clinical trials or have been approved by the FDA (Anker et al., 2008; Grinsztejn et al., 2007; Matsuzaki et al., 2006 and Sato et al., 2006). However, as most of the HIV inhibitors targeting catalytic activity of viral enzyme, strand transfer inhibitors have been reported to interact with human proteins that belong to the same polynucleotidyl transferases family (Dyda et al., 1994), and although they are potent HIV replication inhibitors and highly active on viruses harboring resistance to other antiretroviral classes, these inhibitors rapidly select mutations in integrase gene (Malet et al., 2008 and Shimura et al., 2008). Therefore the development of inhibitors that do not target directly the integrase active site, but its structural organization remains a major challenge.

The Inventors have now designed and evaluated a series of peptides derived from the thumb subdomain of HIV-1 reverse transcriptase using Pep-A as a template. Surprisingly, the Inventors have shown that these peptides exhibit an increased efficiency of the inhibition of reverse transcriptase-polymerase activity of HIV-1 reverse transcriptase compared to Pep-A and, for some of them, further inhibit HIV-1 integrase 3′ processing activity. Particularly, the inventors have shown that among these peptides, the peptides named P_{AW}, P24 and P27 inhibit reverse transcriptase maturation and abolish viral replication without any toxic side-effects. In addition, the peptides named P_{AW}, P16 and P26 also alter the stability and structural organization of the HIV-1 integrase, thus disrupting integrase/partner interactions and preventing its nuclear retention.

Accordingly, in a first aspect, the present invention provides an isolated polypeptide derived from Pep-A peptide (SEQ ID NO: 28) selected amongst

a) peptides consisting of or comprising the amino acid sequence X₁X₂ KWX₃TEX₄X₅PLX₆X₇X₈X₉X₁₀ (SEQ ID NO: 17)

wherein:

X₁ is nothing or G,

X₂ is nothing if X₁ is nothing, and X₂ is T if X₁ is G,

X₃ is L or A

X₄ is W or V,

X₅ is I or A if X₄ is W, and X₅ is W if X₄ is V,

X₆ is nothing or T,

X₇ is nothing if X₆ is nothing, and X₇ is nothing or A if X₆ is T,

X₈ is nothing if X₇ is nothing, and X₈ is E if X₇ is A,

X₉ is A if X₆ is T, and X₉ is nothing if X₆ is nothing, and

X₁₀ is E if X₆ is T, and X₁₀ is nothing if X₆ is nothing,

and

b) peptides consisting of the amino acid sequence SEQ ID NO: 1, or consisting of or comprising an amino acid sequence derived therefrom by the substitution of the amino acid at position 1 of SEQ ID NO: 1 by an alanine (A), or the substitution of one of the amino acids at positions 2, 3, 5, 6 and 8-14 of SEQ ID NO: 1 by an alanine (A) or a glycine (G), or the substitution of the amino acid at position 4 of SEQ ID NO: 1 by a glycine (G) or a valine (V),

wherein said isolated polypeptide inhibits in vitro the HIV-1 Reverse Transcriptase polymerase more efficiently than the peptide Pep-A of amino acid sequence SEQ ID NO: 28.

As used herein, the term “inhibits in vitro the HIV-1 Reverse Transcriptase polymerase more efficiently than the peptide Pep-A of amino acid sequence SEQ ID NO: 28” means that the polypeptide on the invention inhibits in vitro the HIV-1 Reverse Transcriptase polymerase with an inhibition constant (Ki) inferior to that obtained with the peptide Pep-A. This inhibition can be determined by the method described in Restle et al., 1990 or by any other methods, for example by the method described in Example I (see infra).

As used herein, the phrase “substitution of an amino acid” refers to the replacement of an amino acid in a sequence by a different amino acid.

In a preferred embodiment, said polypeptide consists of or comprises the amino acid sequence X₁X₂ KWX₃TEX₄X₅PLX₆X₇X₈X₉X₁₀ (SEQ ID NO: 17) as defined above.

In a more preferred embodiment, said amino acid sequence corresponding to SEQ ID NO: 17 is the amino acid sequence X₁X₂ KWLTEX₃X₄PLX₅X₆X₇X₈X₉ (SEQ ID NO: 34)

wherein:
X₁ is nothing or G,
X₂ is nothing if X_{I} is nothing, and X₂ is T if X₁ is G,

### X₃ is W or V,

### X₄ is I if X₃ is W, and X₄ is W if X₃ is V,

X₅ is nothing or T,
X₆ is nothing if X₅ is nothing, and X₆ is nothing or A if X₅ is T,
X₇ is nothing if X₆ is nothing, and X₇ is E if X₆ is A,
X₈ is A if X₅ is T, and X₈ is nothing if X₅ is nothing, and
X₉ is E if X₅ is T, and X₉ is nothing if X₅ is nothing.

In a particular embodiment, said amino acid sequence corresponding to SEQ ID NO: 17 or SEQ ID NO: 34 is selected from the group consisting of the amino acid sequences SEQ ID NO: 18, 19, 23 to 25 and 27.

In another particular embodiment, said amino acid sequence corresponding to SEQ ID NO: 17 is the amino acid sequence SEQ ID NO: 26.

Said polypeptide can further inhibit in vitro the HIV-1 integrase 3′ processing activity. Non limitative examples of such polypeptides are those consisting of or comprising the amino acid sequences SEQ ID NO: 18, 19 and 24.

As used herein, the term “inhibit in vitro the HIV-1 integrase 3′ processing activity” means that the polypeptide of the invention abolishes the 3′ processing activity of HIV-1 integrase with a concentration between 0.5 μM and 300 μM, preferably about 100 μM. The inhibition of the HIV-1 integrase 3′ processing activity can be determined by the method described in Guiot et al., 2006 or any other methods, for example by the method described in Example III (see infra).

In another embodiment, the amino acid sequence derived from SEQ ID NO: 1 is selected from the group consisting of SEQ ID NO: 2 to SEQ ID NO: 7, and SEQ ID NO: 9 to SEQ ID NO: 15.

Table 1 below shows how the amino acid sequences SEQ ID NO: 1 to SEQ ID NO: 7, SEQ ID NO: 9 to SEQ ID NO: 15, SEQ ID NO: 18 to SEQ ID NO: 19 and SEQ ID NO: 23 to SEQ ID NO: 27 derive from the amino acid sequence SEQ ID NO: 28 (Pep-A) (the substitutions are underlined).

| Pep- | | | | | | | | | | | | | | | | | | SEQ |
|---|---|---|---|---|---|---|---|---|---|---|---|---|---|---|---|---|---|---|
| tide | | | | | | | | | | | | | | | | | | ID |
| name | Amino acid sequences | | | | | | | | | | | | | | | | | NO: |
| | | 1 | 2 | 3 | 4 | 5 | 6 | 7 | 8 | 9 | 10 | 11 | 12 | 13 | 14 | 15 | 16 | |
| Pep-A | R | G | T | K | A | L | T | E | V | I | P | L | T | E | E | A | E | 28 |
| P1 | | G | T | K | A | L | T | E | V | I | P | L | T | E | E | A | E | 1 |
| P2 | | A | T | K | A | L | T | E | V | I | P | L | T | E | E | A | E | 2 |
| P3 | | G | A | K | A | L | T | E | V | I | P | L | T | E | E | A | E | 3 |
| P4 | | G | T | A | A | L | T | E | V | I | P | L | T | E | E | A | E | 4 |
| P5 | | G | T | K | G | L | T | E | V | I | P | L | T | E | E | A | E | 5 |
| P6 | | G | T | K | A | A | T | E | V | I | P | L | T | E | E | A | E | 6 |
| P7 | | G | T | K | A | L | A | E | V | I | P | L | T | E | E | A | E | 7 |
| P9 | | G | T | K | A | L | T | E | A | I | P | L | T | E | E | A | E | 9 |
| P10 | | G | T | K | A | L | T | E | V | A | P | L | T | E | E | A | E | 10 |
| P11 | | G | T | K | A | L | T | E | V | I | A | L | T | E | E | A | E | 11 |
| P12 | | G | T | K | A | L | T | E | V | I | P | A | T | E | E | A | E | 12 |
| P13 | | G | T | K | A | L | T | E | V | I | P | L | A | E | E | A | E | 13 |
| P14 | | G | T | K | A | L | T | E | V | I | P | L | T | A | E | A | E | 14 |
| P15 | | G | T | K | A | L | T | E | V | I | P | L | T | E | A | A | E | 15 |
| | | X₁ | X₂ | K | W | X₃ | T | E | X₄ | X₅ | P | L | X₆ | X₇ | X₈ | X₉ | X₁₀ | 17 |
| | | X₁ | X₂ | K | W | L | T | E | X₃ | X₄ | P | L | X₅ | X₆ | X₇ | X₈ | X₉ | 34 |
| P_{AW} | | G | T | K | W | L | T | E | W | I | P | L | T | A | E | A | E | 18 |
| P16 | | G | T | K | W | L | T | E | V | W | P | L | | | | | | 19 |
| P24 | | G | T | K | W | L | T | E | W | I | P | L | | | | | | 23 |
| P26 | | | | K | W | L | T | E | W | I | P | L | T | A | E | A | E | 24 |
| P27 | | G | T | K | W | L | T | E | W | I | P | L | T | | | A | E | 25 |
| P28 | | G | T | K | W | A | T | E | W | A | P | L | T | A | E | A | E | 26 |
| P29 | | | | K | W | L | T | E | W | I | P | L | T | | | A | E | 27 |

Amino acid sequence SEQ ID NO: 1 is derived from Pep-A in that the arginine residue at N-terminus has been deleted.

Amino acid sequences SEQ ID NO: 2-4, 6, 7 and 9-15 correspond to the amino acid sequence SEQ ID NO: 1 in which, respectively, the amino acids residues at positions 1, 2, 3, 5, 6, 8, 9, 10, 11, 12, 13 and 14 have been substituted by an alanine (A) residue.

Amino acid sequence SEQ ID NO: 5 corresponds to the amino acid sequence SEQ ID NO: 1 in which the amino acid residue at position 4 has been substituted by a glycine (G) residue.

Amino acid sequences SEQ ID NO: 17, 18 and 23-27 derive from the amino acid sequence SEQ ID NO: 1, in that at least the amino acid residues at positions 4 and 8 of the amino acid SEQ ID NO: 1 have been substituted by a tryptophan (W) residue.

In another embodiment of the invention, a cysteine (C) residue is added to the N- or C-terminus of the polypeptide, preferably to the C-terminus, to facilitate the covalent coupling of said polypeptide to a compound such as a chromophore (particularly a fluorophore).

In a particular embodiment of the invention, when the polypeptide of the invention consists of the amino acid sequences SEQ ID NO: 1 or an amino acid sequence derived therefrom or SEQ ID NO: 17 or SEQ ID NO: 34, as defined above, then
said polypeptide can further contain a cysteine residue at the N-terminus or in the case where the polypeptide corresponds to SEQ ID NO: 17 or SEQ ID NO: 34 whereinX₁ and X₂ are respectively G and T as defined above, then the amino acid residue at position 2 of said polypeptide can be substituted by a cysteine residue, and
said polypeptide can further contain a cysteine residue at the C-terminus,
in order to cyclise at least two polypeptides of the invention together.

Preferred examples of such polypeptides are amino acid sequences SEQ ID NO: 35 to 40.

In another embodiment of the invention, one to three amino acid residues of said polypeptide is in D conformation.

In another embodiment of the invention, the N- or C-terminal amino acid of said polypeptide is in beta-conformation.

Preferably, the substitution of an amino acid by an amino acid in D- or beta conformation as defined above should not result in a change of the secondary structure of said polypeptide.

In another aspect, the present invention provides a polypeptide as defined above coupled to a cell delivery agent.

As used herein, the term “coupled” means that the polypeptide of the present invention (named cargo) and the cell delivery agent are non-covalently or covalently linked together. The polypeptide of the present invention can also be indirectly and covalently linked to said cell delivery agent by a cross-linking reagent either to one of the terminal ends of said polypeptide or to a side chain of one of the amino acids of said polypeptide.

As used herein the term “cell delivery agent” refers to a compound capable of delivering or enhancing the delivery of a polypeptide inside the cells in vitro and/or in vivo (i.e., facilitating the cellular uptake of a polypeptide). Non-limitative examples of cell delivery agents are cell-penetrating peptides (CPPs), liposomes, nanoparticles and polycationic molecules (such as cationic lipids). Cell delivery agents are well known to one skilled in the art.

As used herein, the term “cell-penetrating peptide” refers to a peptide of less than 40 amino-acids, preferably less than 30 amino acids, derived from natural or unnatural protein or chimeric sequences, and capable to trigger the movement of the polypeptide of the invention (the cargo) across the cell membrane into the cytoplasm. CPPs can be subdivided into two main classes, the first requiring chemical linkage with the cargo, and the second involving formation of stable, non-covalent complexes. CPPs can be either polycationic, essentially containing clusters of polyarginine in its primary sequence or amphipathic.

CPP-based technologies described so far mainly involve the formation of a covalent conjugate between the cargo and the carrier peptide, which is achieved by chemical cross-linking or by cloning followed by expression of a CPP fusion protein (Nagahara et al, 1998; Gait, 2003; Moulton and Moulton, 2004; Zatsepin et al., 2005, Joliot and Prochiantz 2004; El-Andaloussi et al., 2005; Murriel and Dowdy, 2006). Non limiting examples of CPPs include peptides derived from Tat (Fawell et al., 1994; Vives et al., 1997; Frankel and Pabo, 1998), Penetratin (Derossi et al, 1994), poly-arginine peptide such as the Argg sequence (Wender et al., 2000; Futaki et al., 2001), Transportan, (Pooga et al, 1998), protein derived peptides such as VP22 protein from Herpes Simplex Virus (Elliott & O'Hare, 1997), pVec (Elmquist et al., 2001), Calcitonin-derived peptides (Schmidt et al., 1998; Krauss et al., 2004), antimicrobial peptides Buforin I and SynB (Park et al., 1998), polyproline sweet arrow peptide (Pujals et al., 2006) as well as peptides combining different transduction motifs (Abes et al, 2007) or transduction domains in tandem with protein or oligonucleotide binding domains (Meade and Dowdy, 2008).

Advantageous cell delivery agents are described in International Application WO 2007/069090, such as the peptide vector CADY-2c (SEQ ID NO: 41).

Different chemistries are available for stable or cleavable conjugation involving mainly disulfide or thio-esters linkages. According to the stability and efficiency of the cargo, several parameters need to be considered including the type of linkage chemistry, the nature of the spacer (Gait, 2003; Zatsepin et al., 2005; Snyder and Dowdy, 2005). Conjugation methods offer several advantages for in vivo applications including rationalization, reproducibility of the procedure, together with the control of the stoechiometry of the CPP-cargo.

In a preferred embodiment, the polypeptide of the present invention is non-covalently coupled to a peptide vector, preferably the peptides Pep-1 (SEQ ID NO: 29), Pep-3 (SEQ ID NO: 33) or CADY-2c (SEQ ID NO: 41).

The present invention also provides the use of at least one polypeptide as defined above, preferably a polypeptide comprising or consisting of the amino acid sequences SEQ ID NO: 18 (P_{AW}), SEQ ID NO: 23 (P24) or SEQ ID NO: 25 (P27), for inhibiting, in vitro, ex vivo or in vivo the reverse transcriptase polymerase activity of HIV-1 reverse transcriptase.

The present invention also provides the use of at least one polypeptide selected from the group consisting of the polypeptides comprising or consisting of the amino acid sequences SEQ ID NO: 18, SEQ ID NO: 19 (P16) or SEQ ID NO: 24 (P26), for inhibiting, in vitro, ex vivo or in vivo the HIV-1 integrase 3′ processing activity.

In another aspect, the present invention provides a pharmaceutical composition comprising at least one polypeptide as defined above, preferably selected from the group consisting of the polypeptides comprising or consisting of the amino acid sequences SEQ ID NO: 18, 19, 23, 24 and 25, and at least one pharmaceutically acceptable carrier.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, and 5% human serum albumin. Peptide vectors, liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined hereabove, use thereof in the composition of the present invention is contemplated.

In another aspect, the present invention provides a polypeptide as defined above for use as a medicament, preferably as an antiviral agent.

Said polypeptide is useful for treating or preventing a virus infection, preferably a HIV infection, and more preferably a HIV-1 infection.

As used herein, the term “treating” includes the administration of said polypeptide to a patient who has a virus infection, preferably a HIV infection, more preferably a HIV-1 infection, or a symptom of a virus infection, preferably a HIV infection, more preferably a HIV-1 infection, with the purpose to cure, heal, alleviate, relieve, alter, remedy, ameliorate, improve, or affect the infection, the symptoms of said infections.

The term “preventing” means that the progression of a virus infection, preferably a HIV infection, more preferably a HIV-1 infection, is reduced and/or eliminated, or that the onset of the virus infection, preferably the HIV infection, more preferably a HIV-1 infection, is delayed or eliminated, in a subject having been exposed to (i.e., in contact with) said virus.

In another aspect, the present invention provides a method of treating or preventing a virus infection, preferably a HIV infection, more preferably a HIV-1 infection, in a subject, preferably a human, comprising administering an antiviral effective amount of at least one polypeptide or the pharmaceutical composition as defined above to said subject in need thereof.

When at least two polypeptides of the present invention are used for treating or preventing a virus infection as defined above, then they can be administered separately either simultaneously or sequentially (e.g. as a single composition or different compositions).

The polypeptides or the composition of the invention can be used in combination with one or more antiviral drugs known in the art. When a combination use is conducted, the polypeptides or the composition of the invention and the one or more antiviral drugs can be administered separately, simultaneously or sequentially.

In a preferred embodiment of said combination, the polypeptides or the composition of the invention are used synergistically with the peptide Pep-7 (SEQ ID NO: 32).

The present invention also provides an isolated polynucleotide encoding at least one polypeptide as defined above.

The present invention also provides a recombinant expression cassette, comprising said polynucleotide, under the control of a transcriptional promoter allowing the transcription of said polynucleotide in a host cell.

The recombinant expression cassettes of the invention can be inserted in an appropriate vector, such as a virus, allowing the production of a polypeptide of the invention in a host cell.

Thus the present invention also provides a recombinant vector containing said expression cassette.

The present invention also provides a host cell containing a recombinant expression cassette or a recombinant vector as defined above.

The host cell of the present invention can be a prokaryotic cell (e.g., bacteria) or an eukaryotic cell (e.g., yeast).

The present invention is further illustrated by the additional description which follows, which refers to examples illustrating the preparation and properties of polypeptides of the invention.

### DESCRIPTION OF THE DRAWINGS

FIG. 1 shows the inhibition of the HIV-1 RT polymerase activity by HIV-1 RT derived peptides. HIV-1 reverse transcriptase (40 nM) was incubated with increasing concentrations of peptides, P1 (SEQ ID NO: 1) (), P6 (SEQ ID NO: 6) (φ), P10 (SEQ ID NO: 10) (▪), P11 (SEQ ID NO: 11) (▾) and P_{AW} (SEQ ID NO: 18) (▴), then polymerase reaction was initiated by addition of a mix containing poly(rA). (dT)₁₅ and dTTP substrates. Inhibition constants were extrapolated from Dixon plots.

FIG. 2 shows that HIV-1 reverse transcriptase interacts with P_{AW} peptide (SEQ ID NO: 18) in cultured cells. (A) Interaction between P_{AW} and HIV-1 RT monitored by CNBr-pull-down assay. 30 μg (total protein) per lane were separated on 15% SDS-PAGE and subjected to Western blotting using rabbit anti RT antibody. Lanes correspond to control free beads, P8-, P_{AW}-beads and total proteins loaded on the gel, respectively. (B-G) Interaction between P_{AW} and HIV-1 RT in cellulo was monitored using HeLa cells expressing RT transfected with FITC-P_{AW}/Pep-1 complex formed at a 1/10 molar ratio. HIV-1 RT (Alexa 555 secondary antibody) and FITC-P_{AW} are respectively visualized through a Cy3 and a GFP filter. HeLa cells transfected with free FITC-P_{AW} (B) or complexed with Pep-1 (D). Cultured Hela cells transfected (E) or not (C) with pcDNA3-p66RT. HeLa cells co-transfected with both pcDNA-p66RT and FITC-P_{AW}/Pep-1 at a 1/10 molar ratio. Both RT and P_{AW} display a cytoplasmic localization (D, E) and overlay of the RT, FITC-P_{AW}, shows co-localization of RT and FITC-P_{AW} (G). Nuclear staining with Hoechst dye (F). The RT/P_{AW} colocalisation was analyzed by the 3-D image reconstitution with Imaris 6.0 software of 20 frames from z stacking (G, J). Global view (H), 3D image analysis reveals that RT and P_{AW} co-localize in the cytoplasm at the periphery of the nucleus. 3D analysis of selected cell (arrow) in panel H, with (I) or without (J) nuclear staining. (K) Zoom of the box reported in panel I.

FIG. 3 shows the binding titration of FITC-P_{AW} peptide (SEQ ID NO: 18) to RT and RT-p/t. (A) Titration of FITC-P_{AW} binding to RT (◯), RT:p/t (), p51^{wt}/p66^{F61G} (Δ) or p51^{wt}/p66^{DM} (∇). A fixed 200 nM concentration of FITC-P_{AW} was titrated with increasing concentrations of HIV-1 RTs or RT:p/t. The binding of P_{AW} to RT was monitored by following the quenching of extrinsic P_{AW} fluorescence at 512 nm, upon excitation at 492 nm. (B) Titration of P_{AW} binding to RT:FAM-p/t. A fixed 20 nM concentration of RT:p/t was titrated with increasing concentrations of P_{AW}. The binding of P_{AW} to RT was monitored by following the quenching of extrinsic fluorescence of FAM-labelled p/t at 512 nm, upon excitation at 492 nm. Kd values were calculated using a quadratic equation and correspond to the mean of at least three separate experiments.

FIG. 4 shows the impact P_{AW} peptide (SEQ ID NO: 18) on the binding of primer/template to HIV-1 RT. (A) Titration of fluorescently labelled p/t binding to RT (◯) or RT/P_{AW} () and of FITC-P_{AW}/RT binding to p/t (▴). A fixed 50 nM concentration of fluorescently-labelled p/t was titrated with increasing concentrations of RT or RT/P_{AW}. The binding of p/t to RT was monitored by following the quenching of p/t extrinsic fluorescence at 512 nm, upon excitation at 492 nm. A fixed 100 nM concentration of FITC-P_{AW}/RT complex was titrated by increasing concentrations of p/t (18/36). The binding of FITC-P_{AW}/RT to p/t was monitored by following the quenching of P_{AW} extrinsic fluorescence at 512 nm, upon excitation at 492 nm. Kd values were calculated using a quadratic equation as previously described (Divita et al., 1995b) and correspond to the mean of at least three separate experiments. Kinetics of binding of fluorescently labelled p/t to RT (B) and RT/P_{AW} (C). Typical stopped-flow time courses are reported, where a fixed 20 nM concentration of FAM-labelled p/t was rapidly mixed with 100 nM of RT (B) or RT/P_{AW} (C). Data collection acquisition and analysis were performed using KintAsyst 3 software and kinetics were fitted using a three exponential equation. (D) Secondary plot of the dependence of the fitted pseudo-first order rate constants for the first phase on RT (◯) or RT/P_{AW} () concentration.

FIG. 5 shows the binding of P_{AW} peptide (SEQ ID NO: 18) to heterodimeric RT as monitored by size-exclusion chromatography. (A) Heterodimeric RT (2.3 μM) was incubated in the presence of P_{AW} (10 μM) for 1 h30 at room temperature, then applied onto a gel filtration column and eluted with 200 mM potassium phosphate buffer, pH 7.0. (B) Heterodimeric RT (10 μM) was incubated in the presence of FITC-P_{AW} (SEQ ID NO: 18) (150 μM) for 2 h at room temperature then partially dissociated by 10% acetonitrile for 30 min and analyzed by gel filtration. Proteins were monitored at 280 nm (line 1) and fluorescein-labelled peptide at 492 nm (line 2).

FIG. 6 shows the effect of P_{AW} peptide (SEQ ID NO: 18) on HIV-1 RT-dimerization. (A) Impact of P_{AW} on RT-dimerization. 10 μM of RT was dissociated in the presence of 17% acetonitrile yielding 2 peaks corresponding to p66 and p51 subunits (line 1), then p51 and p66 subunits were diluted in an acetonitrile free buffer and incubated overnight at room temperature in the absence (line 2) or presence of 100 μM P_{AW} (line 3). Kinetics of subunit dimerization 30 min (B) and 2 hrs (C) after dilution in an acetonitrile free buffer. 10 μM of fully dissociated RT were incubated in the presence (line 1) or absence of 100 μM P_{AW} (line 2), then dimerization was induced by dilution in an acetonitrile free buffer and the level of dimer/monomers was monitored at 30 min and 2 hrs by size exclusion chromatography.

FIG. 7 shows that P_{AW} peptide (SEQ ID NO: 18) favours dimerization of the small p51 subunit. HIV-1 p51 (3.5 μM) free (line 2) or incubated with FITC-P_{AW} (20 μM) (line 1) were applied onto a size exclusion chromatography using two HPLC columns in series. Proteins were monitored at 280 nm and fluorescein-labelled P_{AW} at 492 nm (line 3).

FIG. 8 shows that P_{AW} peptide (SEQ ID NO: 18) prevents HIV-1 RT dissociation. (A) P_{AW} associated protection of RT from the acetonitrile dissociation as monitored by size exclusion chromatography. First, HIV-1 RT (8.7 μM) was incubated in the presence (line 1) or absence (line 2) of fluorescently labelled P_{AW} (100 μM) then dissociated by 17% acetonitrile for 30 min at room temperature and applied onto a size exclusion chromatography. (B) Kinetics of RT dissociation induced by acetonitrile. RT (0.5 μM) was dissociated in the presence of 0.8 μM bis-ANS, by adding 10% acetonitrile in the absence (blue line) or presence of 5 μM P_{AW} (red line). The kinetics of dissociation were monitored by following the fluorescence resonance energy transfer between tryptophan of RT and Bis-ANS. Tryptophan excitation was performed at 290 nm and the increase of bis-ANS fluorescence emission at 490 nm was detected through a 420 nm cut-off filter. Data acquisition and analysis were performed using KinetAsyst 3 software (Hi-Tech Scientific, Salisbury, England-UK) and traces were fitted according to a single exponential equation.

FIG. 9 shows the effect of peptide inhibitors on the DNA-binding step and catalytic activity of HIV-1 Integrase (IN). (A) 3′-processing kinetics of IN in the presence of increasing concentrations of P_{AW} (SEQ ID NO: 18). (B) Inhibition of 3′-processing activity by P_{AW} (SEQ ID NO: 18). IN activity was allowed to proceed for 300 min before adding SDS. 3′-processing activities as shown in A and B panels were measured by fluorescence anisotropy. (C) Quantity of formed integrase-DNA complexes in the presence of increasing concentrations of P_{AW} (SEQ ID NO: 18). All the experiments were performed by pre-incubating integrase and the peptide before addition of the fluorescein-labeled DNA substrate. (D) Dissociation of pre-formed integrase-DNA substrate complexes by P_{AW}. 4 nM of fluorescein-labeled DNA substrate was pre-incubated (20 min) with IN (100 nM) in a Tris buffer (20 mM, pH 7.2) containing 50 mM NaCl, 10 mM MgCl2 and 1 mM DTT. P_{AW} was then added (20 μM, 50 μM, 80 μM) and the r value was recorded as a function of time. The relative decrease in the number of complexes was calculated by: (r−rfree)×100/(rt=0−rfree) wherein r and rt=0 correspond to the measured fluorescence anisotropy values accounting for the number of complexes after and before addition of P_{AW}, respectively. rfree corresponds to the fluorescence anisotropy of the free DNA.

FIG. 10 shows the effect of P_{AW} (SEQ ID NO: 18) on IN localisation and IN stability: (A) P_{AW} and P8 (SEQ ID NO: 8) peptides are not able to enter cells alone. When complexed in a non competitive fashion to the cell penetrating peptide Pep-1 (SEQ ID NO: 29), FITC-labelled peptides rapidly localise in the whole cell. (B) In Hela cells, stably expressed HA-IN is mostly located in the nucleus. So does it in the presence of P8-Pep-1. It is not the case in the presence of P_{AW}-Pep-1. (C) HA-IN expressing cells were treated for 0, 30 or 90 min with 100 μg/ml of Cycloheximide, in the presence or not of 1 μM P_{AW}-Pep-1. A noticeable decrease of IN amount is observed with P_{AW}. (D) The latter experiment could be further quantified by normalisation with GAPDH expression, suggesting a twice fold decrease in IN half life (from 30 to 18 min).

FIG. 11 shows the in vivo biodistribution of fluorescent p27 and p16 peptides, 15 min after having been intravenously injected naked (A), or 15 min (B) and 5 hours (C) after having been intravenously injected formulated with CADY-2c/CADY-1S1 (in FIG. 11C, the 3 pictures represent the same mouse). (D) Graph showing the kinetic distribution in mice of radio-labeled peptides p27 (intravenously injected naked or formulated with CADY-2c/CADY-1S1) and p16 (intravenously injected naked or formulated with CADY-2c/CADY-1S1), 24 hrs following injection.

FIG. 12A shows the weight of the mice 1, 5, 10 and 20 days after intravenous injection of peptides p16 and p27 (10 or 20 mg) formulated with CADY-2c/CADY-1S1. (B) Level of TNFα, TNFβ, INFα, IL-6 and IL-12 in mice, 6 hrs after intravenous injection of Poly I:C or peptides p16 and p27 (10 or 20 mg) formulated with CADY-2c/CADY-1 S1.

FIG. 13A shows the number of copies of virus in Hu-PBL-SCID transgenic mice infected with HIV-1 or HIV-1 EFZ (−) virus and treated (20 mg/kg/day) with peptides p27 or p16 formulated with CADY-2c/CADY-1S1, or Efavirenz. (B) Number of HIV-1 copies in mice infected with HIV-1 and treated with peptides p27 or p16 formulated with CADY-2c/CADY-1S1, or Efavirenz at different doses (0, 1, 5, 10 and 20 mg/kg/day).

### EXAMPLE I

### Design and Activity of Peptides Derived from the Thumb Subdomain of HIV-1 Reverse Transcriptase

I.1. Materials and Methods

Materials

Poly(rA)-oligo(dT) and ³H-dTTP (1 μCi/μl) were purchased from Amersham Biosciences (Orsay, France). dTTP was from Roche Molecular Biochemicals, Roche Diagnostics (Meylan, France). MF-membrane (25 mm, 0.45 μm) filters for RT assay were purchased from Millipore (Molshein, France).

Expression and Purification of HIV-1 Reverse Transcriptase Proteins

His-tagged reverse transcriptases (RTs) were expressed and purified as described in Depollier et al. (2005) and Agopian et al. (2007). M15 bacteria (Qiagen Courtaboeuf, France) were separately transformed with all the constructs of p51 and p66 subunits. Cells were grown at 37° C. up to about 0.3 OD₅₉₅, then cultures were cooled to 20° C. and induced overnight with 0.5 mM isopropyl-1-thio-β-D-galactopyranoside. Bacterial cultures expressing His-tagged p66 subunit were mixed with cultures expressing His-tagged p51 subunit to enable dimerization during sonication. For protein isolation and initial purification, the filtered supernatant was applied onto a Hi-Trap chelating column equilibrated with 50 mM sodium phosphate buffer, pH: 7.8, containing 150 mM NaCl supplemented with 50 mM imidazole. The heterodimeric p66/p51 RT was eluted with an imidazole gradient and finally purified by size-exclusion chromatography on a HiLoad 16/60 Superdex 75 column equilibrated with a 50 mM Tris pH: 7.0 buffer containing 1 mM EDTA and 50 mM NaCl. Recombinant untagged-HIV-1 BH₁₀ RT was expressed in E. coli and purified as described in Muller et al. (1989). Highly homogeneous preparations from co-expression of the p66 and p51 subunits were stored in −80° C. in buffer supplemented with 50% glycerol. Protein concentrations were determined at 280 nm using a molar extinction coefficient of 260 450 M⁻¹·cm⁻¹.

Peptide Synthesis

Pep-1 (SEQ ID NO: 29) and P_{AW} (SEQ ID NO: 18) were synthesized using an (fluorenylmethoxy)-carbonyl (Fmoc) continuous (Pionner, Applied Biosystems, Foster city, CA) starting from Fmoc-polyamide linker (PAL)-poly(ethyleneglycol) (PEG)-polystyrene (PS) resin at a 0.05 mmol scale. Peptides were purified by semi-preparative reverse-phase high performance liquid chromatography (RP-HPLC; C18 column Interchrom UP5 WOD/25M Uptisphere 300 5 ODB, 250 mm×21.2 mm) and identified by electrospray mass spectrometry. P_{AW} mM) was coupled to FITC using maleimide-FITC (Molecular Probes. Inc.) (5 mM) through overnight incubation at 4° C. in Phosphate Buffer Saline (PBS: GIBCO BRL). Fluorescently labelled peptide was further purified by RP-HPLC using a C18 reverse-phase HPLC column (Interchrom UP5 HDO/25M Modulo-cart Uptisphere, 250 mm×10 mm) then identified by electrospray mass spectrometry.

Pep-A (SEQ ID NO: 28), P1 peptide (SEQ ID NO: 1), P1 derived peptides (SEQ ID NO: 2-15 and 19-27) and a scrambled peptide (SEQ ID NO: 16) were purchased from GL Biochem, (Shanghai, China) and Genepep, SA. (Prades le Lez, France).

RT-Polymerase Assay

RNA-dependent-DNA RT-polymerase activity was measured in a standard reaction assay using poly(rA)-(dT)₁₅ as template/primer as described in Restle et al. (1990). Ten microliters of reverse transcriptase (RT) at 20 nM was incubated at 37° C. for 30 min with 20 μL of reaction buffer (50 mM Tris, pH 8.0, 80 mM KCl, 6 mM MgCl₂, 5 mM DTT, 0.15 μM poly(rA-dT), 15 μM dTTP, 0.3 μCi 3H-dTTP). For peptide evaluation, HIV-1 RT was incubated with increasing concentrations of peptide inhibitors for 23 hrs, and polymerase reaction was initiated by adding reaction buffer. Reactions were stopped by precipitation of nucleic acids with 5 ml of 20% trichloroacetic acid (TCA) solution for 2 h on ice, then filtered using a multiwell-sample collector (Millipore), and washed with 5% TCA solution. Filters were dried at 55° C. for 30 min and radionucleotide incorporation was determined by liquid scintillation spectrometry.

Antiviral Assay

The anti-HIV activities of the peptides were assayed according to the methods described in Roisin et al. (2004). Phytohemagglutinin-P(PHA-P)-activated peripheral blood mononuclear cells (PBMC) treated by increasing concentrations of peptide (from 100 to 0.1 nM), one hour later, were infected with hundred 50% tissue culture infectious doses (TCID50) per 100,000 cells of the HIV-1-LAI strain (Barre-Sinoussi et al., 1983). This virus was amplified in vitro on PHA-P-activated PBMC. Viral stock was titrated using PHA-P-activated PBMC, and 50% TCID₅₀ were calculated using Kärber's formula (Kärber, 1931). Samples were maintained throughout the culture, and cell supernatants were collected at day 7 post-infection and stored at −20° C. Viral replication was measured by quantifying reverse transcriptase (RT) activity in cell culture supernatants. In parallel, cytotoxicity of the compounds was evaluated in uninfected PHA-P-activated PBMC by colorimetric 3-(4-5 dimethylthiazol-2-yl)2,5diphenyl tetrazolium bromite (MTT) assay on day 7 (as described in Mossmann, 1983). Experiments were performed in triplicate and repeated with another blood donor. Data analyses were performed using SofiMax® Pro 4.6 microcomputer software: percent of inhibition of RT activity or of cell viability were plotted vs concentration and fitted with quadratic curves; 50% effective doses (ED₅₀) and cytotoxic doses (CD₅₀) were calculated.

I.2. Results

Design and Evaluation of HIV-1 Reverse Transcriptase Derived Peptides

A series of peptides have been derived from Pep-A sequence (SEQ ID NO: 28), which is derived from HIV-1 reverse transcriptase. First, P1 peptide (SEQ ID NO: 1) was obtained by removing the N-terminal arginine of Pep-A. Then, additional peptides were generated by performing an alanine scan on P1. P1-derived peptides were evaluated using a standard polymerase RT assay. Ki values extrapolated using Dixon plot analysis are reported in Table 2 below. Reported data correspond to the mean of three separate experiments.

TABLE 2
Inhibition of polymerase activity of HIV-1
RT by Pep-A and P1-derived peptides.

| Pep- | SEQ ID | | |
|---|---|---|---|
| tides | NO: | Amino acid sequence | Ki (μM) |
| Pep-A | 28 | RGTKALTEVIPLTEEAE | 35 ± 5 |
| P1 | 1 | GTKALTEVIPLTEEAE | 7.5 ± 2.3 |
| P2 | 2 | ATKALTEVIPLTEEAE | 28 ± 11 |
| P3 | 3 | GAKALTEVIPLTEEAE | 10.3 ± 2.1 |
| P4 | 4 | GTAALTEVIPLTEEAE | 15 ± 2.9 |
| P5 | 5 | GTKGLTEVIPLTEEAE | 20 ± 3.7 |
| P6 | 6 | GTKAATEVIPLTEEAE | 5.7 ± 2.3 |
| P7 | 7 | GTKALAEVIPLTEEAE | 13.5 ± 2.1 |
| P8 | 8 | GTKALTAVIPLTEEAE | 57 ± 19 |
| P9 | 9 | GTKALTEAIPLTEEAE | 15 ± 7.3 |
| P10 | 10 | GTKALTEVAPLTEEAE | 7.3 ± 2.9 |
| P11 | 11 | GTKALTEVIALTEEAE | 7 ± 1.4 |
| P12 | 12 | GTKALTEVIPATEEAE | 22 ± 3 |
| P13 | 13 | GTKALTEVIPLAEEAE | 10.2 ± 2.5 |
| P14 | 14 | GTKALTEVIPLTAEAE | 14 ± 3 |
| P15 | 15 | GTKALTEVIPLTEAAE | 14 ± 2.2 |
| P_{scramble} | 16 | GAKTETLVIPETELEA | 61 ± 12 |
| P_{AW} | 18 | GTKWLTEWIPLTAEAE | 0.7 ± 0.2 |
| P_{AW}-FITC | | GTKWLTEWIPLTAEAEC-FITC | 2.7 ± 0.7 |

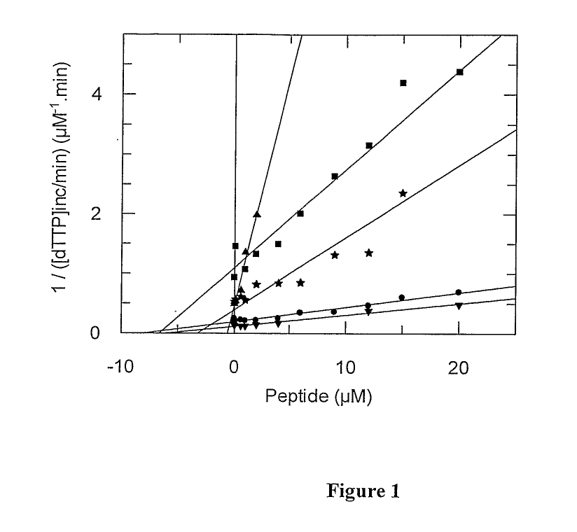

All peptides affected the polymerase activity of reverse transcriptase (RT) in a dose-dependent manner, and four peptides P1 (Ki: 7.5 μM), P6 (Ki: 5.7 μM), P10 (Ki: 7.3 μM) and P11 (Ki: 7.0 μM) possess an inhibition constant lower than 10 μM (FIG. 1).

Pep-A inhibits RT-polymerase activity with an inhibition constant value of 35 μM. Peptide analysis reveals that, surprisingly, removing the Arg¹ residue in Pep-A increases the potency of the peptide (P1) 5-fold. In comparison to P1, mutation of residues Gly¹, Ala⁴, Glu⁷, and Leu¹¹ into alanine significantly affects the potency of the peptide suggesting that the side chains of these residues are required for the interaction with RT. The nature of the side chain of Glu⁷ seems to be a major requirement for the interaction with RT as its substitution by alanine (P8), reduces the efficiency of the peptide 8 fold. Lys³, Thr⁶, Val⁸ and Glu¹⁴ residues have a minor impact as their mutation into alanine only reduces their potency by a factor of 2. Interestingly, the hydrophobic character of Ala⁴ and Val⁸ side chains plays a role in the binding of the peptide to RT and reducing their length affects the potency of the corresponding peptides to inhibit RT 2.7- and 2-fold, respectively.

The two residues Ala⁴ and Val⁸ from P1 were mutated into Trp (W). As shown in FIG. 1, the corresponding peptide P_{AW} (SEQ ID NO: 18) significantly inhibits RT polymerase activity with an inhibition constant (Ki) of 0.7 μM, revealing that mutation of these two residues into Trp improves peptide efficiency 50-fold over Pep-A and 10-fold in comparison to the best lead peptide from the Ala-scan (P6) (see FIG. 1, Table 2).

As the interaction between P_{AW} and RT seems to involve both the N-terminal part and Trp-residues of the peptide, the P_{AW} peptidic-sequence was shortened at the N and/or C-terminal extremities and the positional effect of the Trp was evaluated. All peptides were tested in standard RT assays. Results are represented in Table 3 below.

TABLE 3
Inhibition of polymerase activity of HIV-1 RT by
P_{AW}-derived peptide sequences.

| Pep- | SEQ ID | | |
|---|---|---|---|
| tides | NO: | Amino acid sequence | Ki (μM) |
| P_{AW} | 18 | GTKWLTEWIPLTAEAE | 0.7 ± 0.2 |
| P16 | 19 | GTKWLTEVWPL | 14 ± 4 |
| P17 | 20 | GTKAWTEVWPL | 35 ± 11 |
| P18 | 21 | GTKALTEVIPLT | 53 ± 12 |
| P19 | 22 | GTKAATEVIPLT | 49 ± 9 |
| P24 | 23 | GTKWLTEWIPL | 0.7 ± 0.05 |
| P26 | 24 | KWLTEWIPLTAEAE | 1.8 ± 0.7 |
| P27 | 25 | GTKWLTEWIPLTAE | 0.05 ± 0.01 |
| P28 | 26 | GTKWATEWAPLTAEAE | 2 ± 0.6 |
| P29 | 27 | KWLTEWIPLTAE | 1 ± 0.4 |

Reducing P_{AW} sequence by 2 residues at the N-terminus reduced efficiency 2.5 fold (P26: Ki=1.8±0.7 μM). In contrast, the 5 last residues at the C-terminus of P_{AW} can be removed without affecting its potency to inhibit RT polymerase activity (P24: Ki=0.7±0.05 μM). That P18 does not inhibit RT polymerase activity, confirms that the Trp residues form the major interface with RT. Moving Trp⁸ to position 9 (P16) and both Trp⁴ and Trp⁸ to position 5 and 9 (P17) reduced the efficiency of the corresponding peptides 20-fold (Ki: 14±4 μM) and 50-fold (Ki: 35±11 μM), respectively. Interestingly, removing the last three residues of P_{AW} increases its efficiency 14-fold (P27: Ki=50±0.01 nM).

Results were identical when the peptides of SEQ ID NO: 1-16, 18-27 and 28 further contained a cysteine residue at the C-terminus.

Antiviral Potency of Pep-A Derived Peptides

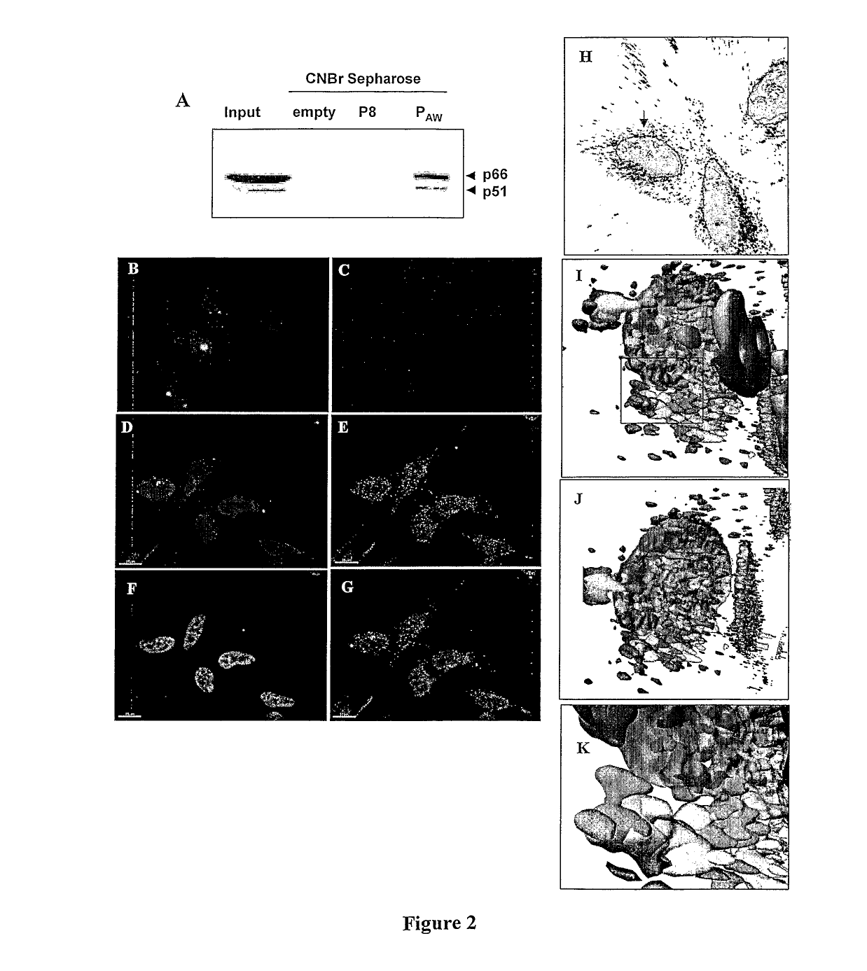

Antiviral activity of the 14 peptides P1, P6, P10, P11, P_{AW}, P16, P17, P18, P19, P24, P26, P27, P28 and P29 (respectively SEQ ID NO: 1, 6, 10, 11, 18-27), was evaluated on PHA-P-activated PBMC infected with HIV-1 LAI. Results (shown in Table 4 below) were reported as 50% efficient concentration (EC₅₀) and selectivity index (SI) corresponding to the ratio between EC₅₀ and the cytotoxic concentration (CC₅₀) inducing 50% death of uninfected PBMCs and relative to Pep-A and P8. In order to avoid any limitation due to the poor ability of peptides to cross cellular membranes, they were associated to the peptide-based nanoparticle delivery system Pep-1 (SEQ ID NO: 29), at a 1/10 molar ratio. Pep-1 has been successfully used for the delivery of peptides and proteins into numerous cell lines as well as in vivo (Gros et al., 2006 and Morris et al., 2001). The inability of free peptides to block viral replication is directly associated to their poor cellular uptake as reported in FIGS. 2B&D for P_{AW}. In contrast when associated with Pep-1, these peptides (P1, P6, P10, P11) block viral proliferation with IC₅₀ values in the low μM range, which correlated with their ability to inhibit HIV-1 RT in vitro (Table 4).

TABLE 4
Antiviral activity of Pep-A, P1 and P1-derived peptides

| Peptides | EC₅₀ (nM) | SI |
|---|---|---|
| P1/Pep-1 | 78.2 | >ND |
| P6/Pep-1 | 170 | >3 |
| P8/Pep-1 | 290 | >3 |
| P10/Pep-1 | 140 | ND |
| P_{AW} | >1000 | ND |
| P_{AW}/Pep-1 | 1.8 | >550 |
| P18/Pep-1 | >1000 | ND |
| P24/Pep-1 | 2.3 | >1200 |
| P26/Pep-1 | >1000 | ND |
| P27 | >1000 | ND |
| P27/Pep-1 | <0.32 | >3100 |

ND: value not determined

In agreement with previous findings no antiviral activity was observed with Pep-A when associated to Pep-1 (Morris et al. 1999a). When complexed with Pep-1, P_{AW} exhibits a marked antiviral activity with an EC₅₀ of 1.8 nM and a therapeutic/selectivity index of about 550. The 44- and 161-fold greater potency of P_{AW} over peptides harbouring mutations at Glu⁷ (P8) or lacking Trp residues (P1) confirms the requirement of these residues for targeting reverse transcriptase both in vitro and in cellulo. P_{AW} constitutes a powerful inhibitor of polymerase activity and possesses a very potent antiviral activity without any toxic effect.

The 5 last residues at the C-terminus of P_{AW} can be removed without affecting its potency to block viral replication (P24: EC₅₀=2.3 nM). Interestingly, removing the last three residues of P_{AW} is also associated with an increase in its antiviral activity with an IC₅₀ lower than 0.32 nM and a therapeutic/selectivity index greater than 3100 (P27).

### EXAMPLE II

### Analysis of the Interaction Between P_{AW} Peptide (SEQ ID NO: 18) and HIV-1 Reverse Transcriptase

II.1. Materials and Methods

Materials

Primer and template oligonucleotides were from MWG Biotech AG, (Ebersberg, Germany). A 19/36-mer DNA/DNA primer/template was used for steady-state fluorescence titration and stopped-flow experiments, with 5′-TCCCTGTTCGGGCGCCACT-3′(SEQ ID NO: 30) for the primer strand and 5′-TGTGGAAAATCTCATGCAGTGGCGCCCGAACAGGGA-3′ (SEQ ID NO: 31) for a template-strand. The sequence of the template strand corresponds to the sequence of the natural primer binding site (PBS) of HIV-1 (Wain-Hobson et al., 1985). The primer was labelled at the 3′-end with 6-carboxyfluorescein (6-FAM) on thymine base. Primer and template oligodeoxynucleotides were separately resuspended in water and diluted to 100 μM in annealing buffer (25 mM Tris pH 7.5 and 50 mM NaCl). Oligonucleotides were mixed together and heated at 95° C. for 3 min, then cooled to room temperature for 1 h.

Steady-State Fluorescence Experiments

Fluorescence experiments were performed in buffer containing 50 mM Tris-HCl, pH 8.0, 50 mM KCl, 10 mM MgCl₂ and 1 mM DTT, at 25° C., using a SPEX-PTI spectrofluorimeter in a 1 cm path-length quartz cuvette, with a band-pass of 2 nm for excitation and emission, respectively. Excitation was performed at 492 nm and emission spectra were recorded from 500 to 600 nm. According fluorescence experiments, a fixed concentration of FAM-labelled (19/36) p/t (50 nM) or of FITC-P_{AW} (200 nM) was titrated with increasing protein concentrations from 5 nM to 1 μM. Data were fitted as described in Agopian et al. (2007) and Rittinger et al. (1995), using a quadratic equation (GraFit, Erithacus Software).

HPLC Size Exclusion Chromatography

Chromatography was performed using one (Phenomenex 53000) or two HPLC columns in series (Phenomenex 53000 followed by Phenomenex 52000; both 7.5 mm×300 mm). Samples containing 3 to 10 μM of RT or p51 were applied onto one or two HLPC columns and eluted with 200 mM potassium phosphate (pH: 7.0) at a flow rate of 0.5 ml·min⁻¹ (5).

Rapid Kinetic Experiments

Binding kinetics of primer-template (p/t) onto HIV-1 RT were performed with a FAM-labelled p/t in buffer containing 50 mM Tris-HCl, pH 8.0, 50 mM KCl, 10 mM MgCl₂ and 1 mM DTT, using a stopped-flow apparatus (Hi-Tech Scientific, Salisbury, England-UK) at 25° C. A fixed concentration of FAM-labeled p/t (20 nM) was rapidly mixed with increasing concentrations of reverse transcriptase (RT) or RT/P_{AW} complex formed at a 1/20 molar ratio (25 to 400 nM). 6-FAM-fluorescence was excited at 492 nm and emission detected through a filter with a cut-off at 530 nm. Data acquisition and analysis were performed using KinetAsyst 3 software (Hi-Tech Scientific, Salisbury, England-UK) and traces were fitted according to a three exponential equation, as previously described in Agopian et al. (2007). The rate constant for the first phase (k₊₁ and k₋ᵣ), corresponding to the formation of a RT/P_{AW}-p/t collision complex, was extrapolated from the slope and the intercept with the y axis of the plot of k_{obs1} vs RT concentrations. The k₂ (k₊₂+k₂) and k₃ (k₊₃+k₃) rate constants for the second and third phases corresponding to conformational changes of preformed RT/P_{AW}-p/t complex were directly obtained from the three exponential fitting.

Dissociation kinetics of HIV-1 RT were monitored by using 4,4′-Bis(1-anilinonaphthalene 8-sulfonate (bis-ANS) as extrinsic probe. Changes in bis-ANS fluorescence provide a good signal to probe variation in the exposure of the hydrophobic regions associated to RT dissociation in a time-dependent manner. 0.5 μM of RT was dissociated in presence of 0.8 μM of bis-ANS, by adding 10% acetonitrile in the absence or in the presence of 10 μM P_{AW}. Kinetics of dissociation were monitored by following fluorescence resonance energy transfer between tryptophan residues of RT and bis-ANS. Excitation of RT-Trp residues was performed at 290 nm and the increase of bis-ANS fluorescence emission at 490 nm was detected through a 420 nm cut-off filter. Data acquisition and analysis were performed using KinetAsyst 3 software (Hi-Tech Scientific, Salisbury, England-UK) and traces were fitted according to a single exponential equation.

Cell Culture, Transfection and Indirect Immunofluorescence Microscopy

HeLa cells were cultured in Dulbecco's modified Eagle's medium (DMEM) supplemented with 10% fetal calf serum at 37° C. in a humidified atmosphere containing 5% CO₂.). H9 cell lines (ATCC number: HTB-176) were stably transfected with pNL4.3 V−R+plasmid (AIDS Research Reference Reagent Program, National Institutes of Health (NIH), USA) and therefore expressed in a constitutive manner all HIV-1 proteins but Env encoded proteins. They were cultured in RPMI-1640 medium supplemented with 2 mM glutamine, 10% (w/v) foetal calf serum (FCS), 1% antibiotics (streptomycin 10 000 mg/ml, penicillin, 10 000 IU/ml), as well as G418 1 mg/ml. Cells were grown on glass coverslips to 75% confluency, then transfected with pcDNA3-p66RT plasmid (this plasmid contains the 66 subunit of HIV-1 Reverse Transcriptase cloned in pcDNA3.1 (+) vector (Invitrogen, USA), using HindIII and BamHI restriction site) using Lipofectamine 2000 reagent according to manufacturer' instructions (Invitrogen). Hela cells were subsequently cultured for 32 h, before incubation with FITC-P_{AW} or FITC-P_{AW}/Pep-1 (complex obtained at a molar ratio 1/10) for 1 h. Coverslips were extensively rinsed with PBS and cells were fixed in 4% paraformaldehyde for 10 mM and permeabilized in 0.2% Triton. After saturation in PBS supplemented with bovine serum albumine 1% for 1 h, cells were incubated overnight with monoclonal 8C4 anti-HIV-1 RT antibody (AIDS Research Reference Reagent Program, NIH) (diluted 1:100 in PBS-BSA 1%), followed by Alexa-555 anti-mouse (Molecular Probes).

Immunofluorescence detection of HIV-1 RT and FITC-P_{AW} was performed by epifluorescence microscopy using a PL APO 1.4 oil PH3 objective on a LEICA DMRA 1999 microscope. 3D reconstitution of the 20 frames realized from z stacking was performed using Imaris 6.0 software.

For RT pull down experiments, P_{AW} (SEQ ID NO: 18) was incubated with 500 μl of activated CNBr-activated Sepharose 4B beads at 4° C. overnight. After centrifugation, supernatants were removed and the beads were incubated with Glycine pH.8.0 for 2 hrs at 4° C. with gentle stirring. The beads were then washed with 0.1 M sodium acetate buffer (pH 4.0), then 0.5 M Bicarbonate buffer, and finally in PBS, three times each. The peptide bound to the beads were then saturated for 30 min in PBS/BSA 0.1% and then incubated for 1 h at 4° C. with equal amounts of H9 cells lysed for 30 mM on ice in Lysis buffer (Tris 20 mM, pH 7.2, NaCl 400 mM, EDTA 1 mM, DTT 1 mM, Protease inhibitors EDTA free) and sonicated 2×5 sec. at 20%. Beads were washed with Lysis buffer then twice with PBS and the bound proteins were finally separated on 15% SDS-PAGE gel and analyzed by Western Blotting using monoclonal 8C4 anti-HIV-1 RT antibody.

II.2. Results

P_{AW} Peptide Interacts with HIV-1 RT in a Cellular Context

The ability of P_{AW} (SEQ ID NO: 18) to form stable complexes with HIV-1 RT expressed in cells was investigated by pull-down experiments. The peptides P_{AW} and P8 (SEQ ID NO: 8) covalently associated with CNBr sepharose beads, was incubated in the presence of cell lysates of H9 cells expressing Gag-Pol gene products of HIV-1. Analysis of the presence of reverse transcriptiase (RT) by Western blotting revealed than only P_{AW} was able to form stable complex with RT in a cellular context and to retain RT on beads (FIG. 2A). In contrast, no RT was associated to free or P8-beads. To confirm that P_{AW} targets HIV-1 RT in a cellular context, it has been further investigated its localization in cells transfected by a plasmid expressing HIV-1 p66 (pcDNA3-p66RT). As shown in FIG. 2B, fluorescently-labelled peptide (FITC-P_{AW}) poorly enters cultured HeLa cells. In contrast, when complexed at a molar 1110 ratio with the Pep-1 peptide-based nanoparticle delivery system, FITC-P_{AW} rapidly (less than 1 h) enters cells (FIG. 2D). P_{AW} localizes mainly in the cytoplasm with a peri-nuclear accumulation and partially co-localizes with HIV-1 RT as determined by indirect immunofluorescence (FIGS. 2E and 2G). The RT/P_{AW} interaction in cellulo was further characterized using 3D reconstitution of frames from z stacking. 3D image analysis reveals that P_{AW} does not enter the nucleus and co-localize with RT at the periphery of the nucleus (FIGS. 2G and 2H).

Binding of P_{AW} Peptide to the Dimeric Form of HIV-1 RT

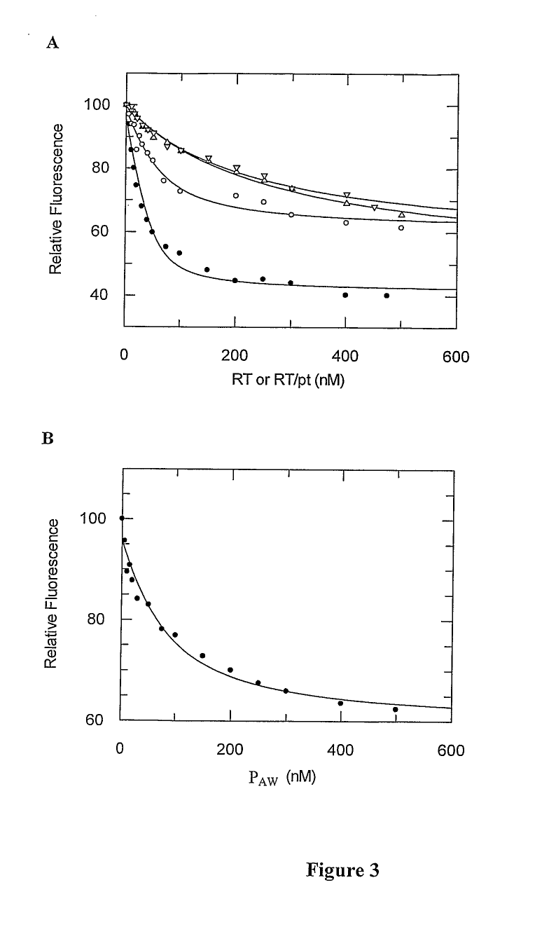

To further understand the mechanism through which P_{AW} inhibits RT, it has been investigated its potency to interact with the dimeric form of HIV-1 RT in the absence or presence of DNA/DNA p/t. The binding of P_{AW} to RT was monitored using a fluorescently labelled peptide (FITC-P_{AW}). It has been first evaluated the impact of P_{AW} labelling on the C-terminal cysteine with an FITC-probe on its ability to inhibit RT polymerase activity. As reported in Table 2 above, FITC-P_{AW} blocks RT polymerase activity with a Ki of 2.7±0.7 μM, 3.8-fold greater than for P_{AW}, suggesting that labelling has only a minor effect on P_{AW} inhibitory property. As reported in FIG. 3A, upon binding to the dimeric form of RT, the fluorescence of FITC-P_{AW} was quenched by 39% and analysis of the titration curves revealed that P_{AW} tightly binds heterodimeric RT with a dissociation constant (Kd) of 33±10 nM. When RT is first incubated with DNA/DNA p/t (18/36 mer), the quenching of FITC-P_{AW} fluorescence associated with its binding was of 57% and the affinity of FITC-P_{AW} for RT increased 5-fold (Kd: 7.1±2.8 nM), suggesting that the presence of p/t on RT facilitates the binding of P_{AW}. The association of unlabelled P_{AW} to RT was also evaluated by monitoring changes in fluorescently labelled p/t bound to RT (FIG. 3B). Binding of P_{AW} results in a 39% quenching of fluorescence and a Kd value of 40±18 nM was estimated from the titration binding curve. The 5.6-fold lower Kd of labelled FITC-P_{AW} over unlabelled peptide, suggests that the dye contacts RT and stabilizes the peptide within its binding site.

As both Trp²⁴ and Phe⁶¹ located on the fingers domain of p66 subunit have been reported to be involved in the control of p/t binding and in the dynamics of the thumb-fingers subdomain interactions (Agopian et al., 2007 and Fisher et al., 2002 and 2003), the binding of P_{AW} on RT harbouring single phe^{61Gly} and double Phe^{61Gly} and Trp^{24Gly} mutations on the p66 subunit were then evaluated. In comparison to wild type RT, the affinity of P_{AW} was reduced 6-fold (Kd: 207±62 nM) for p66^{F61Gly}/p51^{wt} and 4.5-fold (Kd: 149±38 nM) for p66^{DM}/p51^{wt}(FIG. 3A).

Effect of P_{AW} Peptide on Primer/Template Binding to HIV-1 RT

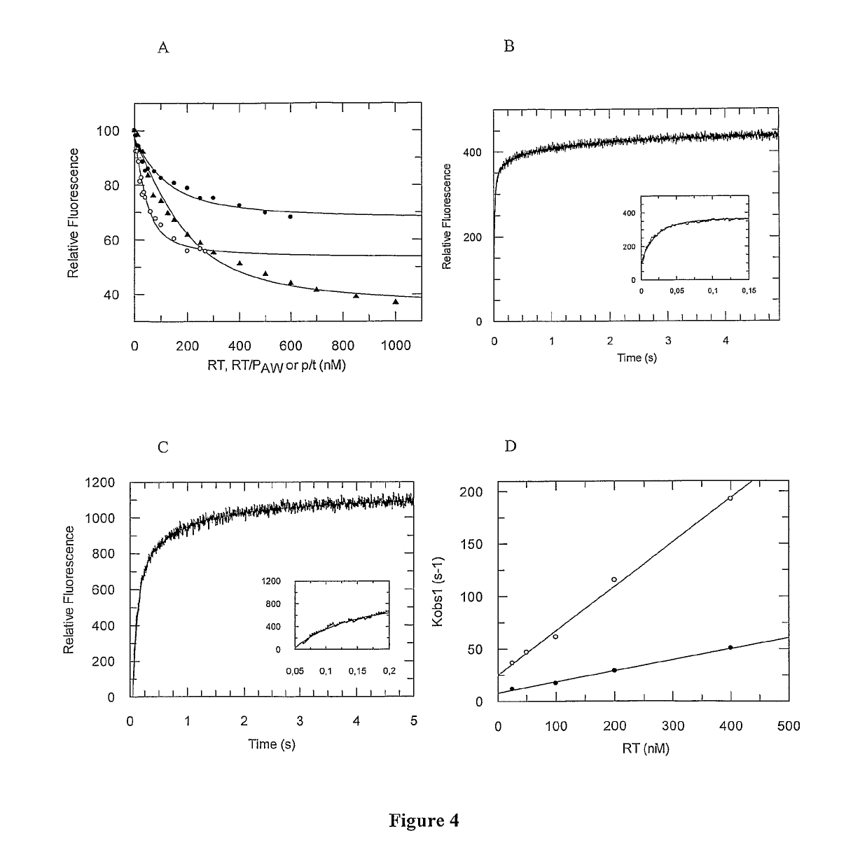

The impact of P_{AW} peptide on the ability of HIV-1 RT to bind p/t was then investigated at both steady-state and pre-steady state levels using a 19/36 mer p/t labelled at the 3′-end of the primer with FAM-derivative as previously described (Agopian et al., 2007 and Rittinger et al., 1995). Results are shown in FIG. 4A. The presence of a saturating concentration of P_{AW} (10 μM) decreases the affinity of fluorescently labelled-p/t for RT 4.5-fold with a Kd value of 99±40 nM in comparison to 22±5 nM obtained in the absence of P_{AW}. The binding of unlabeled p/t induces a 42% change in the fluorescence of P_{AW}-FITC pre-bound to RT and leads to a similar Kd value of 66.5±19 nM (FIG. 4A). These results suggest that p/t interacts close to P_{AW} binding site on RT, inducing a change in the orientation of FITC linked to P_{AW}, but does not share the same binding site as already reported for Pep-A (Morris et al., 1999a).

RT-p/t pre-steady-state binding kinetics follow a three-step mechanism in the presence or in the absence of P_{AW}, including a rapid diffusion controlled second order step leading to the formation of the RT-p/t collision complex, followed by two slow, concentration-independent, conformational changes (Agopian et al., 2007). The plot of the pseudo-first order rate constant for the initial association of the p/t with RT against RT-concentration is linear. In the absence of P_{AW}, k₊₁ and k₁ rate constant values of 4.23.10⁸ M⁻¹·s⁻¹ and 29.9 s⁻¹ were calculated from the slope and the intercept with the y axis of the graph (FIGS. 4B and 4C). Analysis of the second and third slow phases yielded rate constants of k₂=5.8 s⁻¹ and k₃=0.76 s⁻¹ for RT. The presence of P_{AW} does not alter the overall Kd1 for the initial formation of the RT-p/t complex as both the “on” (k₊₁=1.05.10⁸ M⁻¹·s⁻¹) and the “off” (k_{—1}=7.9 s⁻¹) rates of the first step are decreased by about 4-fold. In contrast, the presence of P_{AW} on RT significantly reduced the rate constants of the slow conformational steps (k₂=1.99 s⁻¹ and k₃=0.22 s⁻¹), affecting the proper binding of the p/t (FIGS. 4B & 4C).

Effect of P_{PW} on the Stability and Dimerization of HIV-1 RT

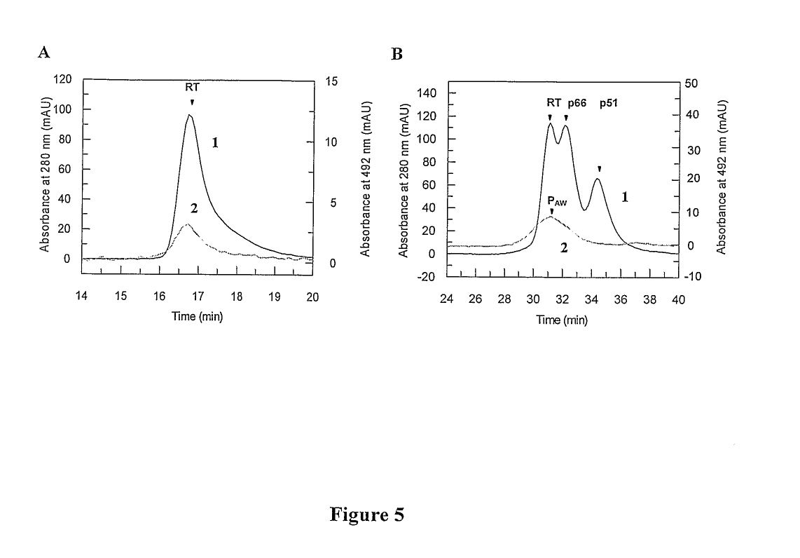

The impact of P_{AW} on the stability and formation of heterodimeric-RT was investigated by size-exclusion chromatography as previously described (Divita et al., 1995a). Results are represented in FIG. 5A. Heterodimeric-RT incubated or not in the presence of an excess of P_{AW} (100 μM) for 1 h30 at room temperature, is fully dimeric and eluted as a single peak at 16.7 min. The interaction of P_{AW} with RT was monitored by size-exclusion chromatography using HIV-1 RT pre-incubated with FITC-P_{AW}. Chromatography analysis reveals that FITC-P_{AW} co-elutes with heterodimeric RT in a single peak at 16.7 min (FIG. 5A), demonstrating that P_{AW} binds heterodimeric RT and does not induce RT dissociation. The ability of P_{AW} to interact with p66 or p51 monomeric forms was then evaluated. Experiments performed with a partially dissociated RT/P_{AW} (50%) complex by 10% acetonitrile, show that P_{AW} remains associated only with the dimeric fraction of RT, and does not bind monomeric p66 or p51 subunits, which are eluted at 17.5 min and 18.2 min, respectively (FIG. 5B).

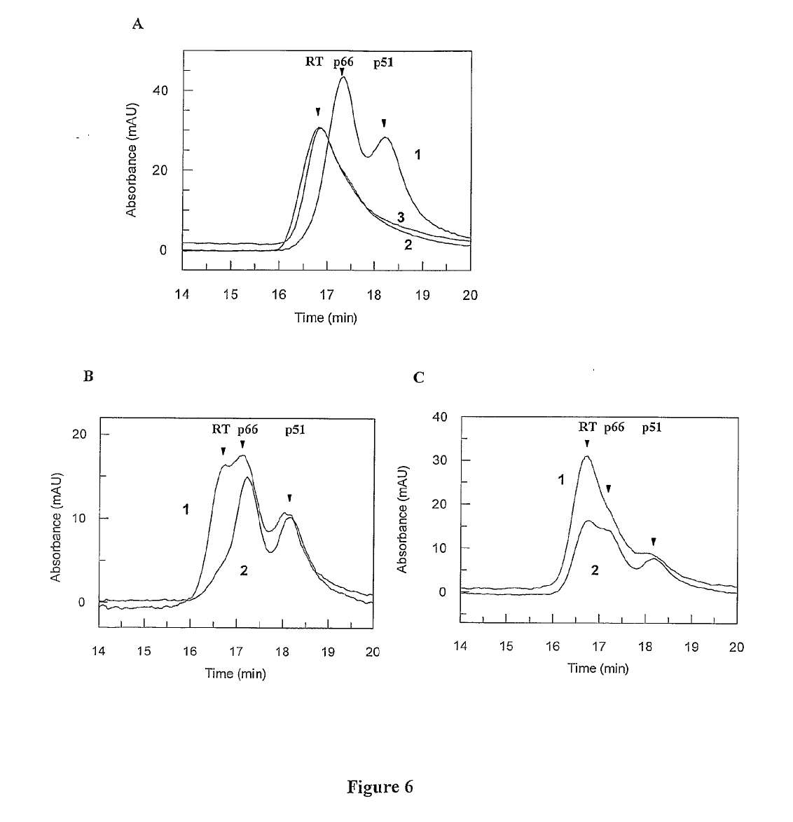

The ability of P_{AW} to prevent HIV-1 RT-dimerization was then investigated. Dissociation of RT was achieved at room temperature with 17% acetonitrile, and then association of the subunits was induced by a 10-fold dilution of the sample in an acetonitrile-free buffer in the absence or presence of 100 μM of P_{AW}. At this concentration (1.7%) of acetonitrile no dissociation of RT could be detected. As shown in FIG. 6A, heterodimeric RT was fully re-associated 5 hrs after dilution in an acetonitrile-free buffer, both in the absence or in the presence of P_{AW} (100 μM), indicating that P_{AW} does not block RT dimerization (FIG. 6A). The impact of P_{AW} was further investigated on the kinetics of RT-dimerization. The level of dimeric RT was evaluated 30 min and 2 hrs after dilution in free acetonitrile buffer by size-exclusion chromatography (FIGS. 6B and 6C). In the presence of P_{AW} 21% and 59% of dimeric RT was quantified after 30 min and 2 hrs respectively (FIG. 6B). In comparison only 16% (30 min) and 29% (2 hrs) of dimeric RT were detected in the absence of peptide (FIG. 6C), suggesting that the presence of P_{AW} favours the kinetics of RT dimerization.

P_{AW} Peptide Favours Dimerization of the Small P51 Subunit

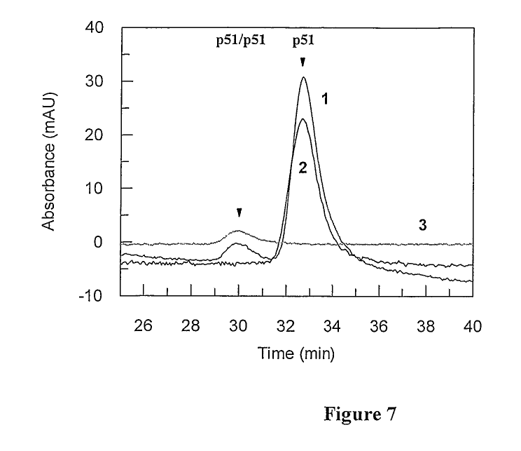

P51 subunits are mainly monomeric and dissociation constants for p51/p51 homodimer have been reported to be either in the μM (Venezia et al., 2006) or mM (Restle et al., 1990) range depending on the technology used to quantify the interactions. The ability of P_{AW} to favour p51/p51 dimerization has been investigated by size exclusion chromatography, using two HPLC columns in series. Experiments were performed at a p51 concentration of 3.5 μM at which it is entirely monomeric and elutes as a single peak at 32.7 min. Monomeric p51 (3.5 μM) was incubated in the presence of FITC-labelled P_{AW} (20 μM) for 1 h at room temperature then analysed by size exclusion chromatography. Results are shown in FIG. 7. In the presence of fluorescently-labelled P_{AW} 4.6% of p51 are dimeric and associated to P_{AW}, suggesting that P_{AW} promotes p51/p51 homodimer and only p51/p51 homodimer.

P_{AW} Peptide Prevents HIV-1 RT Dissociation

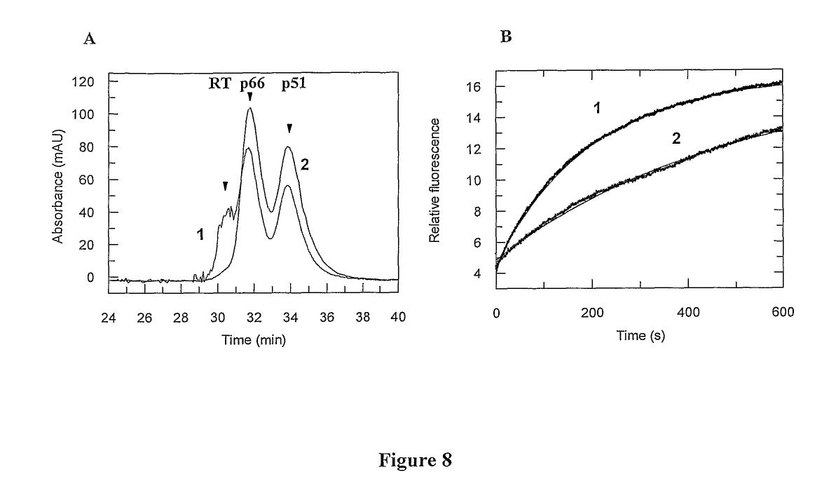

The impact of P_{AW} on HIV-1 RT stability and dissociation were investigated at the steady state level by size exclusion chromatography and at the pre-steady state level by stopped-flow rapid kinetics. HIV-1 RT was preincubated in the presence of 100 μM P_{AW}, for 2 hrs, prior dissociation with 17% or 10% of acetonitrile, and the level of dimeric form was then assessed by size exclusion chromatography and the rate of dissociation by pre-steady-state kinetics. As reported in FIG. 8A, the presence of P_{AW} protects RT from the acetonitrile dissociation as 17% remains dimeric whereas “free” RT is completely dissociated with 17% acetonitrile.

The protection by P_{AW} of acetonitrile-associated RT-dissociation was further investigated by monitoring pre-steady-state dissociation kinetics of HIV-1 RT, using bis-ANS as an extrinsic probe (Divita et al., 1995c). Binding of bis-ANS to dissociated RT resulted in a large increase in the fluorescence of the probe due to non covalent interactions of bis-ANS to exposed hydrophobic surfaces on RT subunits, therefore providing a good signal for following RT dissociation in a time-dependent manner. Experiments were performed by adding bis-ANS to HIV-1 RT prior dissociation of the enzyme by 10% acetonitrile and monitoring FRET between exposed Trp of RT and Bis-ANS. As reported in FIG. 8B, the kinetics of increased ANS-fluorescence upon dissociation of RT in the absence of P_{AW} follow a single-exponential reaction, with a dissociation rate constant k_{dis} of 5.30±0.01 s⁻¹, which is reduced 3.8-fold (k_{dis}=1.42±0.007 s⁻¹), when RT is incubated with P_{AW}.

### EXAMPLE III

### Analysis of the Interaction Between P_{AW} Peptide (SEQ ID NO: 18) and HIV-1 Integrase (IN)

III-1. Materials and Methods

Materials

Peptides were prepared as described in Example I.1 above.

Western Blot Analysis and Antibodies

The protein-containing supernatants were separated by SDS/PAGE, transferred onto Nitrocellulose membranes, and revealed by immunoblotting with the following antibodies as indicated: polyclonal anti-Integrase, or monoclonal anti-HA (HA.11; Sigma).

Cell Culture

Adherent fibroblastic HeLa cell line stably expressing HA-tagged Integrase (HelIN cells) (Mousnier et al., 2007) were cultured in DMEM supplemented with 2 mM glutamine, 1% antibiotics (streptomycin 10 000 mg/ml, penicillin, 10 000 IU/ml), 10% (w/v) foetal calf serum (FCS), and 1 μg/ml Puromycin (SIGMA). Modified ATCC H9 cell lines were cultured and prepared as described in Example II.1 All cells lines were maintained at 37° C. in a humidified atmosphere containing 5% CO2.

Integrase Pull Downs Using CNBR Sepharose Activated Beads

Peptides were resuspended in buffer A (1 mg/ml), sonicated 4 min, and incubated with 500 μl (gel volume) of activated CNBr-activated Sepharose 4B sepharose© beads at 4° C. overnight. After centrifugation, supernatants were removed and the beads were incubated with Glycine pH.8 for 2 hours at 4° C. with gentle stirring. The sedimented Sepharose beads were then washed in 0.1 M Sodium Acetate buffer (pH 4), 0.5 M Bicarbonate buffer, and finally in PBS, three times each. The peptides bound to the beads were then saturated for 30 minutes in PBS BSA 0.1% and then incubated for 1 h at 4° C. with equal amounts of cell lysate (H9 or Hela-IN) prealably lysed for 30 min on ice in Lysis buffer (Tris 20 mM, pH 7.2, NaCl 400 mM, EDTA 1 mM, DTT 1 mM, Protease inhibitors EDTA free (Roche Diagnostics) and sonicated twice for 5 sec. at 20%. Beads were washed once in Lysis buffer, and twice in PBS. They were finally resuspended in Laemli blue, migrated on 15% SDS-PAGE gels and analysed by Western Blotting.

Cellular Localization Experiments and Pep-1-Mediated Transfection

For carrier peptide Pep-1 (SEQ ID NO: 29) mediated delivery of P_{AW} peptide, stock solutions of Pep-1-P_{AW} complex were formed by incubation of P_{AW} (SEQ ID NO: 18) with the carrier peptide at a molecular ratio of 1/10 in PBS for 30 min at 37° C. They were then diluted in DMEM to the desired concentration.

Cells were grown on acid-treated glass coverslips to 60% confluence. Once rinsed with PBS, cells were overlaid with preformed complexes and incubated for 40 minutes at 37° C. Cells were then rinsed twice with PBS and fixed 15 mM at Room Temperature) (RT° in Paraformaldehyde 4%, and washed three times in PBS+2% BSA. They were then permeabilised by incubation 10 min RT° in PBS+0.5% Triton X-100, washed again 3 times in PBS+2% BSA. After blocking 20 min in the same solution, the coverslips were placed in a humid chamber, overlaid with 75 μl of primary antibody against Integrase, and incubated for 2 h at 37° C. Coverslips were then rinced once with PBS and incubated with 75 μl of anti rabbit antibody diluted 1/10000 in PBS. After immobilisation on the cover using Prolong Gold antifade reagent, cellular localization of Integrase as well as FITC-labelled peptides was monitored by fluorescence microscopy, using a PL APO 1.4 oil PH3 objective on a LEICA DMRA 1999 microscope. For suspension cell lines, cells were harvested by centrifugation and resuspended directly with the preformed complex solutions for 5 min and then the level of foetal calf serum was adjusted to 10%.

Cycloheximide Treatment

Cycloheximide treatment was done as described in Mousnier et al. (2007). Cells were incubated at 37° C. with 100 mg/ml cycloheximide for various periods of time prior to washing in PBS and lysis in Laemmli sample buffer. Protein content of the total cell lysates was quantified (Bio-Rad protein assay kit). Equal amounts of total cellular proteins were resolved by SDS/PAGE and analyzed by Western blot with the indicated antibodies.

Fluorescence Anisotropy Experiments

Steady-state fluorescence anisotropy parameter (r) was recorded on a Beacon 2000 instrument (PanVera, Madison, USA), in a cell thermostatically held at 25° C. for DNA-binding assay or 37° C. for activity test (the sample volume was typically 200 μL).

Integrase-Peptide Interaction

Fluorescein-labeled peptides (40 nM) were mixed with varying concentrations of integrase in 20 mM Tris pH 7.2, 50 mM NaCl, 10 mM MgCl₂, and the r values were recorded. Fluorescence intensities of peptides did not significantly changed upon addition of integrase.

DNA-Binding of Integrase and 3′-Processing Assay

The simultaneous measurement of integrase-DNA interaction and subsequent 3′-processing activity by steady-state fluorescence anisotropy was performed as described in Guiot et al. (2006). 4 nM of fluorescein-labeled double stranded oligonucleotide (21-mer oligonucleotide mimicking the U5 LTR extremity with fluorescein attached on the 3′-extremity of the processed end) was added to a preincubated mixture containing IN (100 nM) and increasing concentration of unlabeled peptides in a Tris buffer (20 mM, pH 7.2) containing 50 mM NaCl, 10 mM MgCl₂ and 1 mM DTT. r values obtained upon addition of integrase (r_{complex}) were used to measure the IN-DNA interaction as IN binding to the fluorescein-labeled oligonucleotide significantly increases the fluorescence anisotropy. After the initial DNA-binding step (performed at 25° C.), the 3′-processing activity was measured at 37° C. by fixed-time experiments: The reaction was stopped at varying times by adding SDS (0.25% final) which disrupts all IN-DNA complexes in the sample. The IN-mediated cleavage of the terminal GT dinucleotide (labeled by fluorescein on the 3′-end) leads to a significant decrease of r as compared to the r value corresponding to the fluorescein-labeled unprocessed DNA substrate. The fraction of released dinucleotide is calculated by using Eq. (1):

$${F}_{dinu} = \frac{{r}_{NP} - r}{{r}_{NP} - {r}_{dinu}} \qquad ( 1 )$$

wherein r_{NP} and r_{dinu} are the anisotropy values corresponding to the unprocessed double-stranded DNA substrate and GT dinucleotide, respectively.

For dissociation experiments, IN-DNA substrate complexes were pre-formed at 25° C. before addition of peptides. The peptide-mediated dissociation of complexes was then studied at the same temperature by measuring the decrease in the r value as a function of time.

III.2. Results

P_{AW} Peptide Interacts with HIV-1 Integrase

P_{AW} peptide (SEQ ID NO: 18) was first assessed on its ability to form stable complex with HIV-1 integrase either recombinant or expressed in cells, by pull-down and steady-state fluorescence anisotropy experiments. P_{AW} covalently associated to CNBr sepharose beads, was incubated with either recombinant IN or cell lysate of H9 and Hela cells, expressing GAG-POL gene products of HIV-1, or HA-tagged IN, respectively. Beads were then extensively rinsed and presence of IN was detected by western blotting.

P_{AW} was able to interact with cellular HA-tagged IN. P_{AW} beads retained cellular IN when expressed at low level in H9 cells. As a control, the reverse transcriptase (RT) dimerization inhibitor Pep-7 (Morris et al., 1999b) does not bind IN, suggesting a specific impact of the Trp residues in the P_{AW} context.

The direct interaction between P_{AW} and integrase was further assessed by steady-state fluorescence anisotropy using fluorescein-labeled peptides. The steady-state anisotropy value (r) of labelled-P_{AW} was measured in the presence of increasing concentrations of IN. Upon addition of integrase, the r value of P_{AW} increased (Δr=0.060). Fitting of the titration binding curve leads to an apparent dissociation constant for the P_{AW}/IN complex of Kdₐₚₚ 400 nM in perfect agreement with the tight P_{AW}/IN interaction obtained in the pull down experiments. The impact of the P_{AW} peptide onto IN oligomerization was also assessed as described in Deprez et al. (2001) and no effect of P_{AW} on the IN oligomer integrity was observed.

P_{AW} Peptide Inhibits IN 3′ Processing Activity In Vitro

The potency of P_{AW} to inhibit 3′ processing activity of IN was evaluated using a steady-state fluorescence anisotropy in vitro assay as described in Guiot et al. (2006). A fluorescently labelled DNA was used to monitor the binding of IN to its DNA substrate and the subsequent 3′ processing activity, both event being associated with changes on the anisotropy parameters.

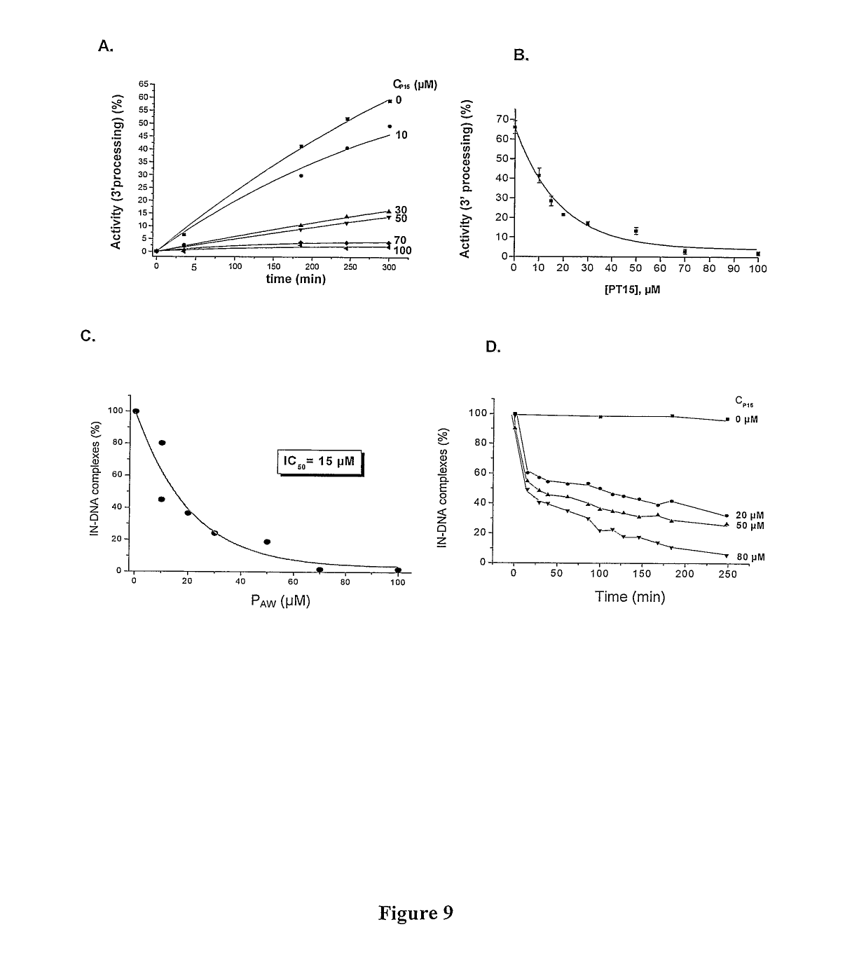

It was first validated that P_{AW} does not interact with DNA, then the 3′-processing kinetics of IN were measured in the absence or presence of a fixed P_{AW} concentration of 100 μM. As reported in FIG. 9A, the 3′-processing activity was totally abolished with 100 μM P_{AW}, suggesting a correlation between the propensity of P_{AW} to interact with IN and its inhibitory effect on the catalytic activity. The P_{AW} mediated inhibition of the 3′-processing activity is peptide concentration dependent and an IC50_{3′-proc} value of 12.7 μM was calculated (FIG. 9B).

Results were identical when P_{AW} peptide further contained a cysteine residue at the C-terminus.

P_{AW} Induces Dissociation of Preformed in/DNA Complexes

To get insight into the mechanism of inhibition, the ability of P_{AW} to prevent IN-DNA recognition was measured. As reported in FIG. 9C, the number of IN/DNA complexes decreased as a function of P_{AW} concentration, with an IC50_{DNA-binding} value of 15.2 μM, compatible with the value found for the inhibition of the 3′ processing activity. This suggests that P_{AW} does not inhibit the catalytic process by itself but most likely inhibits the DNA-binding step as previously reported for small chemical compounds such as styrylquinoline derivatives (Deprez et al., 2004). However, in contrast to styryquinoline compounds which were not active on preformed IN-DNA complexes (Deprez et al., 2004), P_{AW} does not prevent association of IN to its DNA substrate, but also effectively dissociates preformed complexes in a concentration dependent manner with similar efficiency than the one obtained when P_{AW} and IN were pre-incubated before adding the DNA substrate (FIG. 9D). Interestingly, the apparent dissociation constant measured for the formation of P_{AW}-IN complex (400 nM) was significantly below the IC50_{3′P} and IC50_{DNA-binding} values. Two alternative hypothesis can account for this apparent discrepancy: (i) as the IN/P_{AW} interaction was probed using a fluorescein-labelled counterpart of P_{AW}, it could be possible that the fluorescein moiety stabilizes the complex, (ii) the high affinity site is not directly related to the competitive inhibition mode.

In order to verify the influence of the fluorescent probe attached to P_{AW} on the peptide-integrase interaction, a standard gel-electrophoresis procedure (as described in Smolov et al., 2006) was carried out to examine the IN 3′-processing activity as a function of either unlabeled or fluorescein-labeled P_{AW} concentration. The fluorescein-labeled P_{AW} was characterized by an IC50_{3′-proc} value of 10 μM, in the same range that the value found for the unlabeled P_{AW}, suggesting that the fluorescein does not influence the binding or inhibition properties of P_{AW}. Thus, the discrepancy between the apparent affinity for the IN-P_{AW} complex (submicromolar) as compared to the IC₅₀ value (in the 10-15 μM range) most likely accounts for the presence of peptide binding site on the integrase that differs from its active site and indirectly influence 3′-processing activity.

Insight into the Cellular Mechanism of P_{AW}

The fact that P_{AW} blocks viral production with a subnamolar EC₅₀ cannot only be explained by inhibition of IN enzymatic activity (IC50_{3′-proc}: 15.2 μM) and is more correlated to the tight binding of the peptide to IN. In order to address that point, it has been investigated the mechanism by which this peptide alters IN activities at the cellular level.

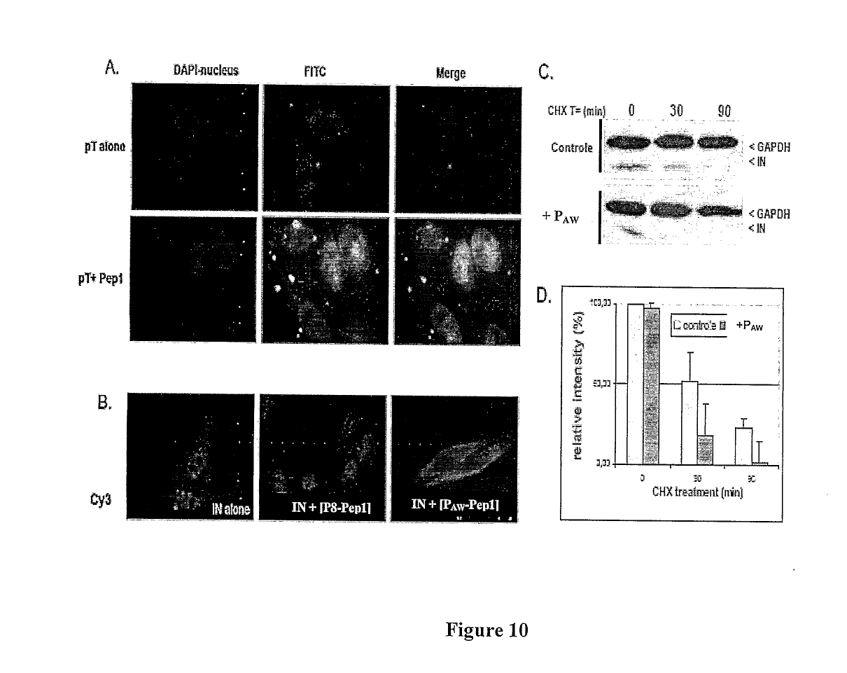

Hela cells stably expressing Ha-tagged IN were used to analyze the cellular behaviour of IN on P_{AW}-treated cells. As previously reported, the PIC-mediated nuclear import of IN constitutes a major step in the infection cycle and IN mainly localizes in the nucleus (Bukrinsky et al., 1992; Depienne et al., 2001 and Piller et al., 2003). As reported in FIG. 10, in the absence of P_{AW}, Ha-tagged IN localizes in the nucleus of Hela cells. In contrast, when cells were treated with P_{AW} (1 μM), IN did not localizes in the nucleus, suggesting that IN interaction with P_{AW} within the cell, prevents its nuclear localization and/or induces its accumulation/retention in the cytoplasm. The impact of P_{AW} has been further investigated in High Content Screening (HCS) using a higher number of cells. Statistic analysis of the cytoplasmic retention of IN has revealed that in the absence of P_{AW} more than 90% of the integrase located in the nucleus, in contrast to only 35% when cell are treated with P_{AW}.

It has also been demonstrated that the cytoplasm retention of IN is dependent on the concentration of P_{AW} used and does not occur with either the free Pep-1 (SEQ ID NO: 29) particles or P8 peptide (SEQ ID NO: 8)/Pep-1 complexes (FIG. 10B).

P_{AW} Alters the Stability of In

In the presence of P_{AW}, IN was mostly detected in the cytoplasm. It has been investigated to what extend P_{AW} was acting on pre-existing integrase or rather that on newly-formed protein.

It was previously shown that HA-tagged IN is a very unstable protein when overexpressed in Hela cells, with a half life of 23 min, as estimated by cycloheximide treatment (Mousnier et al., 2007). Therefore the stability of IN in the presence of P_{AW} was investigated. Cells were treated with Cycloheximide for 0, 30 or 90 minutes, in the absence of the presence of 1 μM of P_{AW} (FIG. 10D). In the absence of P_{AW}, IN a half-life was estimated at about 30 min, which is consistent with the results previously described (Mousnier et al., 2007). In contrast, in the presence of the peptide, IN half-life was reduced by about 2-fold, (estimated 17 min).

Taken altogether, these results suggest that P_{AW} binding induces a structural destabilisation of IN, which alters interaction with partners and promotes its degradations, resulting in an apparent nuclear delocalisation.

### EXAMPLE IV

### P_{AW}, P16 and P27 Block Resistant Strains and HIV Clades

IV.1. Materials and Methods

Materials

P_{AW} (SEQ ID NO: 18), P16 (SEQ ID NO: 19), P27 (SEQ ID NO: 25) and Pep-1 (SEQ ID NO: 29) peptides were prepared as described in Example I.1 above. Pep-3 peptide (SEQ ID NO: 33) was prepared as described in Morris et al., 2007. Pep-7 peptide (SEQ ID NO: 32) was prepared as described in Morris et al., 1999b.

HIV strains BH10, 1650, RF, 2914, NDK, 2165, HIV-1 215Y, HIV-1 67N, 70R, 215F, 219Q, HIV-1 74V, HIV-1 N119/181C and HIV-1 41L, 74V, 106A, 215Y were obtained from the National Institutes of Health, USA (AIDS Research Reference Reagent Program).

Methods

The anti-HIV activities of the peptides were assayed according to the methods described in Roisin et al. (2004) (see also Example I.1 above).

IV.2. Results

P_{AW} and P27 Block Replication of HIV-1 Clades and Combining Pep-7/P27 Improves Efficiency

P27 peptide was associated to the peptide based nanoparticle delivery system (Pep-1 or Pep-3) at a ratio 20/1. For combining experiments, equi-molar concentrations of Pep-7 and P27 were associated with the carrier Pep-3 at a molar ratio of 1/20 and applied onto anti HIV evaluation. Results are shown in Table 5 below. Data are the averages of three separate experiments.

TABLE 5

| HIV-1 | | | IC50 (nM) | | |
|---|---|---|---|---|---|
| Strain | Clade | Country | P_{AW} | P27 | P27/Pep-7 |
| BH10 | | | 4.6 | 1.4 | 0.3 |
| 1650 | A | France | 4.1 | 0.8 | 0.8 |
| RP | B | Haiti/USA | 3.5 | 1.5 | 0.4 |
| 2914 | C | USA | 2.4 | 0.2 | 0.3 |
| NDK | D | Zaire | 1.5 | 0.3 | 0.09 |
| 2165 | E | Asia | 5.1 | 2.6 | 0.7 |

P_{AW} and P27 block replication of resistance strains and combining Pep-7/P27 improves antiviral potency

Results are shown in Table 6 below:

| | | IC50 (nM) | | |
|---|---|---|---|---|
| RT Genotype | Phenotype | P_{AW} | P27 | P27/Pep-7 |
| HIV-1 215Y | AZT-resistant | 4.1 | 2.7 | 2.1 |
| HIV-1 67N, | AZT-resistant | 3.7 | 1.5 | 0.9 |
| 70R, 215F, 219Q | | | | |
| HIV-1 74V | resistant to ddI and ddC | 5.2 | 3.8 | 1.3 |
| HIV-1 | resistant to nevapirine | 5.1 | 2.0 | 1.8 |
| N119/181C | and non nucleoside RT | | | |
| | inhibitors | | | |
| HIV-1 41L, 74V, | resistant to AZT, ddI, | 4.8 | 4.9 | 1.4 |
| 106A, 215Y | nevapirine; non | | | |
| | nucleoside RT inhibitors | | | |

P16 Blocks Replication of HIV-1 Clades and Combining P16/Pep-7 Improves Efficiency

P16 peptide was associated to the peptide based nanoparticle delivery system (Pep-1 or Pep-3) at a ratio 20/1. For combining experiments, equimolar concentrations of Pep-7 and P16 were associated with the carrier Pep-3 at a molar ratio of 1/20 and applied onto anti HIV evaluation. Results are shown in Table 7 below. Data are the averages of three separate experiments.

TABLE 7

| | | | IC50 (nM) | IC50 (nM) |
|---|---|---|---|---|
| Strain | Clade | Country | P16 | P16/Pep-7 |
| BH10 | A | France | 5.9 | 1.2 |
| 1650 | B | Haiti/USA | 7.5 | 0.5 |
| RF | C | USA | 10.9 | 1.9 |
| 2914 | D | Zaire | 1.7 | 2.0 |
| NDK | E | Asia | 2.9 | 0.2 |
| 2165 | F | France | 2.7 | 1.8 |

P16 and P16/Pep-7 Block Replication of Resistance Strains

Results are shown in Table 8 below:

TABLE 8

| | | IC50 (nM) | |
|---|---|---|---|
| RT Genotype | Phenotype | P27 | P27/Pep-7 |
| HIV-1 215Y | AZT-resistant | 3.9 | 2.1 |
| HIV-1 67N, | AZT-resistant | 5.2 | 4.0 |
| 70R, 215F, 219Q | | | |
| HIV-1 74V | resistant to ddI and ddC | 4.9 | 2.6 |
| HIV-1 | resistant to nevapirine | 6.3 | 3.1 |
| N119/181C | and non nucleoside RT | | |
| | inhibitors | | |
| HIV-1 41L, 74V, | resistant to AZT, ddI, | 10.5 | 1.9 |
| 106A, 215Y | nevapirine; non | | |
| | nucleoside RT inhibitors | | |

### EXAMPLE V

### P_{AW} and Derived-Peptides Therefrom Alter IN Structure and Function

V.1. Materials and Methods

Materials

P_{aw} (SEQ ID NO: 18) P16 (SEQ ID NO: 19), P17 (SEQ ID NO: 20), P18 (SEQ ID NO: 21), P24 (SEQ ID NO: 23), P26 (SEQ ID NO: 24) and P27 (SEQ ID NO: 25) were prepared as described in Example I.1 above.

Methods

Effect of the different peptides on the integrase (IN) activity, DNA binding and IN/DNA dissociation were evaluated as described in Example III.1 above, using fluorescence polarization and anisotropy measurements. Binding constant of the peptide for IN was determined by steady state fluorescence and anisotropy.

Dissociation of IN/LEDGF complex was measured by fluorescence resonance energy transfer (AlphaScreen® technology) and immunoprecipitation experiments:
Amplified Luminescent Proximity Homogeneous Assay (ALPHA): when the proteins (IN and LEDGF), linked to acceptor and donor beads, interact together (i.e., are into close proximity), a photosignal (a high amplified signal with output in the 520-560 nm range) is detected; when the proteins are not in close proximity, the reactive oxygen decays and there is no detectable signal generated;
immunoprecipitation (IP) was performed using polyclonal anti LEDGF antibodies (purchased from Santa Cruz Biotechnology, USA) and polyclonal HA antibodies (purchased from Eurogentec, Belgium). Cell line expression HA-tagged integrase was using for the experiment in the presence of increasing concentrations of peptide inhibitors.

V.2. Results

Results are shown in Table 9 below:

| | | | EC50 (μM) | | | | | | | |
|---|---|---|---|---|---|---|---|---|---|---|
| Pep- | | | | | IN/DNA | | IN/DNA | | IN/LEDGF | |
| tides | Kd (nM) | | 3′processing | | binding | | dissociation | | dissociation | |
| P_{AW} | 230 | nM | 12 | μM | 10 | μM | 15 | μM | 2 | μM |
| P16 | 120 | nM | 10 | μM | 8 | μM | 11 | μM | 0.8 | μM |
| P24 | 350 | nM | 33 | μM | 35 | μM | 21 | μM | 13 | μM |
| P26 | 320 | nM | 28 | μM | 54 | μM | 67 | μM | >50 | μM |

| | | | | | | | | | |
|---|---|---|---|---|---|---|---|---|---|
| P27 | >100 | μM | >100 | μM | >100 | μM | >100 | μM | NO |

| | | | | | |
|---|---|---|---|---|---|
| P18 | NO | NO | NO | NO | NO |
| P17 | NO | NO | NO | NO | NO |

P_{AW} and P16 tightly interacted with integrase and therefore induce conformational changes than prevent and even dissociate interactions between IN and its DNA substrate, and a cellular partner (LEDGF).

### EXAMPLE VI

### Evaluation of Cyclic P_{AW}, P16 and P27 Peptides

VI.1. Materials and Methods

Materials

Peptide cyclization was performed via disulfide linkages S—S bond. P_{AW} (SEQ ID NO: 18), P16 (SEQ ID NO: 19) and P27 (SEQ ID NO: 25) where respectively cyclised by either adding to the peptides sequences a cysteine residue at the N-terminus and a cysteine residue at the C-terminus, or by substituting the amino acid residue at position 2 by a cysteine residue and by adding a cysteine residue at the C-terminus:

Peptides Sequences:

| | |
|---|---|
| PAW-Cl: CGTKWLTEWIPLTAEAEC | (SEQ ID NO: 35) |
| PAW-C2: GCKWLTEWIPLTAEAEC | (SEQ ID NO: 36) |
| P16C1: CGTKWLTEVWPLC | (SEQ ID NO: 37) |
| P16C2: GCKWLTEVWPLC | (SEQ ID NO: 38) |
| P27C1: CGTKWLTEWIPLTAEC | (SEQ ID NO: 39) |
| P27C2: GCKWLTEWIPLTAEC | (SEQ ID NO: 40) |

Methods

Effect of the different cyclized peptides on RT polymerase activity was determined as described in Example I above. The effects on IN activity, DNA binding and IN/DNA dissociation were evaluated as described in Example III above, using fluorescence polarization and anisotropy measurements. Binding constant of the peptide for IN was determined by steady state fluorescence and anisotropy.

VI.2. Results

Results are shown in Table 10 below:

| Pep- | Ki RT | | Kd IN | | Ki IN | | EC50 | |
|---|---|---|---|---|---|---|---|---|
| tides | polymerase | | binding | | 3′processing | | HIV-1 lai | |
| PAW | 0.7 | μM | 230 | nM | 12 | μM | 1.8 | nM |
| PAW-C1 | 0.2 | μM | 290 | nM | 21 | μM | 1.2 | nM |
| PAW-C2 | 1.4 | μM | 120 | nM | 8 | μM | 2.7 | nM |
| P16 | 14.4 | μM | 120 | nM | 10 | μM | 5.9 | nM |
| P16C1 | 32.1 | μM | 90 | nM | 4.3 | μM | 2.5 | nM |
| P27 | 0.05 | μM | >100 | μM | >100 | μM | 0.21 | nM |
| P27C1 | 0.042 | μM | >100 | μM | >100 | μM | 0.17 | nM |

Cyclized P_{AW}, P16 and P27 peptides respectively inhibit RT polymerase and IN 3′ processing activity and alter the stability of IN in vitro.

### EXAMPLE VII

### In Vivo Biodistribution of P27 and P16 Peptides

VII.1. Materials and Methods

In order to evaluate the potency of the peptides p27 (SEQ ID NO: 25) and p16 (SEQ ID NO: 19) in vivo, it was analyzed their ability to be systemically administrated by intravenous injection in mice (BALB/c; 6 to 8 week-old female mice). Peptides were either radio-labeled at the N-terminal amino acid or fluorescently labeled with Alexa® Fluo700 probe. 10 μg of each peptide were formulated with the peptide CADY-2c (GLWRALWRALWRSLWKKKRKV-Cya; SEQ ID NO: 41, described in International Application WO 2007/069090) in water at a ratio 1/20, then particles were coated with 3% of CADY-1S1 (Cholesterol-CAₚ-GLWRALWRLLRSLWRLLWKA; SEQ ID No: 42) as described in International Application WO 2007/069090. Particles were injected intravenously (100 μl) and kinetic of peptide distribution were followed by fluorescence live imaging. 24 hr after injection, the level of peptides in the different tissues were quantified by fluorescence and radioactivity.

VII.2. Results

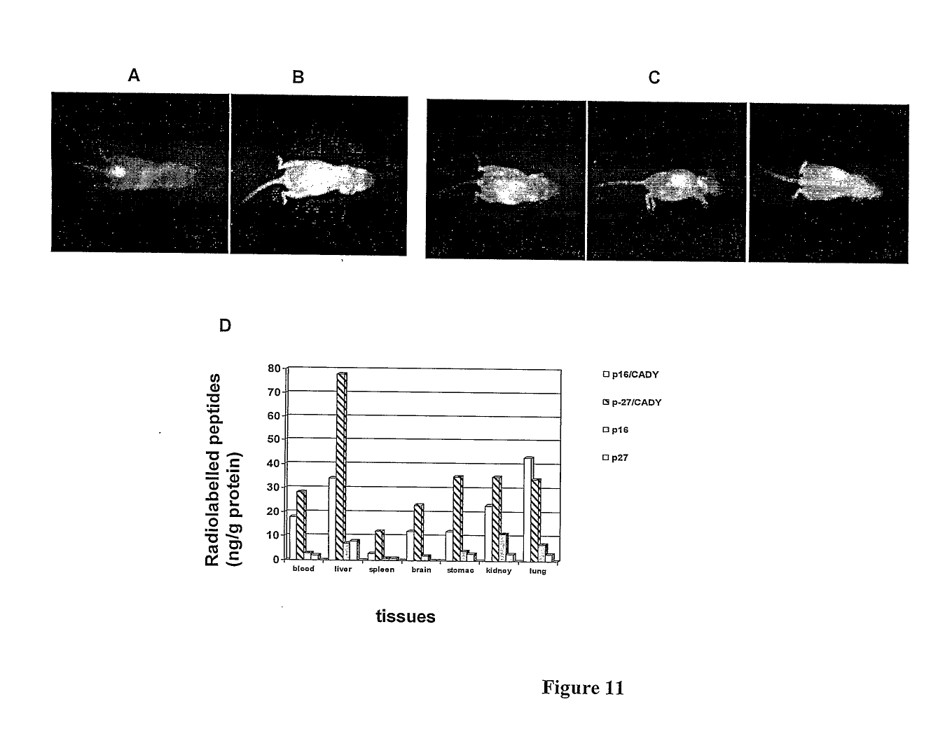

Results are shown in FIG. 11. Naked p27 and p16 peptides were rapidly eliminated and rapidly accumulated mainly in the bladder after 15 min (FIG. 11A), with a half-life of less than 1 hour. However, when formulated with CADY-2c/CADY-1S1, formulation favored a rapid distribution of p27 and p16 peptides throughout the body within the first 15 min following injection (FIG. 11B). Peptides were found to access all tissues and distribution was optimal after 5 hr (FIG. 11C). After 24 hr, peptide accumulation was observed in liver, brain, stomach, kidney, lung and large amount of peptide remained in the blood circulation, suggesting that formulation increase the bioavailability of both peptide inhibitors p27 and p16 (FIG. 11D).

### EXAMPLE VIII

### In Vivo Toxicity of P27 and P16 Peptides

VIII.1. Materials and Methods

In order to evaluate the in vivo toxicity of the peptides p27 (SEQ ID NO: 25) and p16 (SEQ ID NO: 19), different concentration (5, 10, 20 mg) of peptides were intravenously injected into mice (BALB/c; 6 to 8 week-old female mice) and both cytokine/immune response and changes in animal weight (5 animal pr group) were analyzed. Peptides were formulated with the peptide vector CADY-2c (see above) in water at a ratio 1/20, then particles were coated with 3% of CADY-1S1 (see above), as described in International Application WO 2007/069090. Particles were injected intravenously (1000) every 2 days for 1 week (D, 3, 5 and 7). Animal body weight was measured every 2 days for 20 days and level of mouse cytokine tumor necrosis factor-alpha and -beta (TNFα, TNFβ), interferon-alpha (INFα) and interleukin 6 (IL-6) and 12 (IL-12) were quantified 6 hrs after injection of any formulation, using sandwich ELISA assay kit (DB Bioscience). The immunostimulant Poly I:C (polyinosinic:polycytidylic acid) (200 μg) was used as a control.

VIII.2. Results

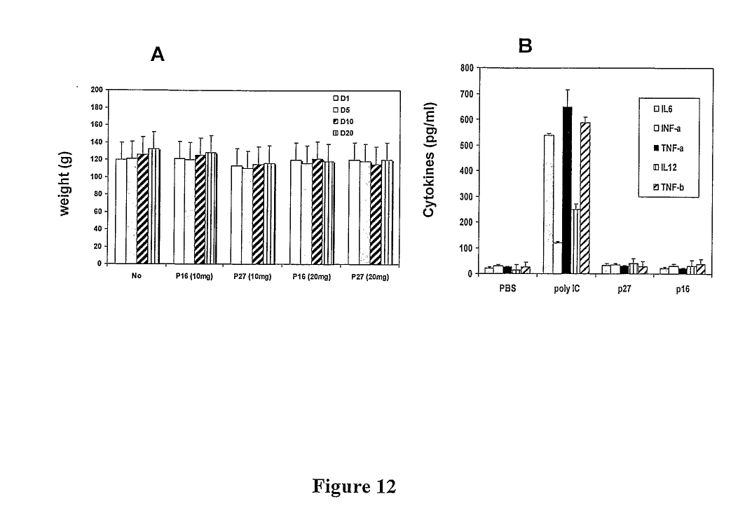

Results are shown in FIG. 12. No changes in the animal's weight and behavior was observed whatever the peptide concentration used (FIG. 12A). No activation of inflammatory cytokine and interferon response was observed whatever the formulation in contrast to injection of Poly IC, which increases by 10 fold the level of cytokines (FIG. 12B).

### EXAMPLE IX

### P27 and P16 Peptides Block HIV Propagation In Vivo

IX.1. Materials and Methods

The potency of the peptides p27 (SEQ ID NO: 25) and p16 (SEQ ID NO: 19) was assessed in vivo following intravenous challenge of Hu-PBL-SCID transgenic mice (in which human (hu) peripheral blood leukocytes (PBLs) from healthy Epstein-Barr virus (EBV)-seropositive donors are injected into severe combined immunodeficiency (SCID) mice; Mosier et al., 1988). The mice (6 to 8 week-old male mice, 5 animals per group) were infected with wild type HIV 1 virus or efavirenz-resistance HIV strain harbouring K103N and N341V mutations on reverse transcriptase. Infected mice were treated with peptides formulated with the peptide vector CADY-2c (see above) in water at a ratio 1/20, and then particles were coated with 3% of CADY-1S1 (see above), as described in International Application WO 2007/069090. Particles were injected intravenously (100 μl) every 2 days for 1 week, then HIV-1 copy were quantified by quantitative PCR. Efavirenz (a non-nucleoside reverse transcriptase inhibitor; EFZ) was used a positive drug control.

IX.2. Results

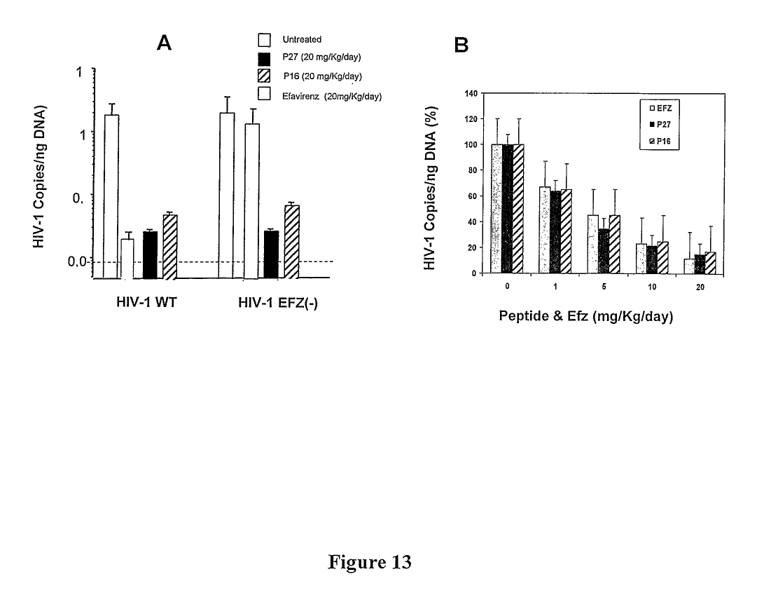

Similarly to Efavirenz, at 20 mg/kg both peptides block HIV propagation in vivo and level of HIV 1 DNA copies in spenocyte extracts remains negligible (FIG. 13A). Data correspond to the average of a cohort of 5 animals per group. In contrast, when challenged with EFZ resistance strain, p27 and p16 reduced viral propagation by 72% and 58%, respectively whereas no effect was observed with EFZ. In vivo data demonstrated than p27 and p16 peptides when associated to CDAY-2c/CADY-1S1 block virus propagation in vivo. Dose response was performed ranging peptide concentrations from 1 to 20 mg/kg/day. The efficiency of the peptide is as potent as EFZ on wild type virus, with 50% reduction in the number of HIV-1 copies obtained with 5 mg/kg/day peptide (FIG. 13B).

### REFERENCES

Agopian, A. et al. (2007) J. Mol. Biol. 373, 127-140.
Anker, M. and Corales, R. B. (2008) Expert Opin. Investig. Drugs 17, 97-103
Barre-Sinoussi, F. et al. (1983) Science 220, 868-871
Bukrinsky, M. I. et al. (1992) Proc. Natl. Acad. Sci. USA 89, 6580-6584
Cabodevilla, J. F. et al. (2001) Eur. J. Biochem. 268, 1163-1172
Camarasa, M. J. et al. (2006) Antiviral Res. 71, 260-267
Cherepanov, P. et al. (2003) J. Biol. Chem. 278, 372-81
Craigie, R. (2001) J. Biol. Chem. 276, 23213-6
De Clercq, E. (2007) Nat. Rev. Drug Discov. 6, 1001-1018
De Coupade, C. et al. (2005) Biochem. J. 390, 407-18
Depienne, C. et al. (2000) Experimental Cell Research 260, 387-395
Depienne, C. et al. (2001) J. Biol. Chem. 276, 18102-18107
Depollier, J. et al. (2005) Biochemistry 44, 1909-1918
Deprez, E. et al. (2001) Proc. Natl. Acad. Sci. USA 98, 10090-10095.
Deprez, E. et al. (2004) Mol. Pharmacol. 65, 85-98
Derossi, D. et al. (1996) J. Biol. Chem. 271, 18188-18193
Di Marzo Veronese, F. et al. (1986) Science 231, 1289-1291
Divita, G. et al. (1994) J. Biol. Chem. 269, 13080-13083
Divita, G. et al. (1995a) J. Biol. Chem. 270, 28642-28646
Divita, G. et al. (1995b) J. Mol. Biol. 245, 508-521
Divita, G. et al. (1995c) Biochemistry 34, 16337-16346
Dyda, F. et al. (1994) Science 266, 1981-6
Elliott, G. and O'Hare, P. (1997) Cell 88, 223-233
Farnet, C. M. et al. (1997) Cell 88, 483-92.
Figueiredo, A. et al. (2006) PLoS pathogens 2, el 19
Fisher, T. S., and Prasad, V. R. (2002) J. Biol. Chem. 277, 22345-22352
Fisher, T. S., Darden, T., and Prasad, V. R. (2003) J. Mol. Biol. 325, 443-459
Ghosh, M. et al. (1996) Biochemistry 35, 8553-8562
Grinsztejn, B. et al. (2007) Lancet 369, 1261-9
Gros, E. et al. (2006) Biochimic et biophysica acta 1758, 384-393
Guiot, E. et al. (2006) 281, J. Biol. Chem. 281, 22707-22719
Hsiou, Y. et al. (1996) Structure 4, 853-860
Huang, H. et al. (1998) Science 282, 1669-1675
Huang, S. C. et al. (1992) Biochem. Biophys. Res. Commun. 184, 986-992
Jacobo-Molina, A. et al. (1993) Proc. Natl. Acad. Sci. USA 90, 6320-6324
Kärber. (1931) Naunyn-Schmiedebergs Archiv für experimentelle Pathologie and Pharmakologie 162, 480-483
Kohlstaedt, L. A. et al. (1992) Science 256, 1783-1790
Kokryakov, V. N. et al. (1993) FEBS Lett. 327, 231-236
Lightfoote, M. M. et al. (1986) J. Virol. 60, 771-775
Malet, I. et al. (2008) Agents Chemother. 52, 1351-1358
Matsuzaki, Y. et al. (2006) Presented at the XIIIth CROI Denver, USA, February 5-9 2006
Morris, M. C. et al. (1999a) Biochemistry 38, 15097-15103
Morris, M. C. et al. (1999b) J. Biol. Chem. 274, 24941-24946
Morris, M. C. et al. (2001) Nat. Biotech. 19, 1173-1176
Morris, M. C. et al. (2007) Nucleic Acids Res. 35, e49
Mosier, D. E., et al. (1988) Nature (London) 335, 256-259
Mossmann T. (1983) J. Immunol. Methods 65, 55-63
Mousnier, A. et al. (2007) Proc. Natl. Acad. Sci. USA 104, 13615-20.
Muller, B. et al. (1989) J. Biol. Chem. 264, 13975-13978
Patel, P. H. et al. (1995) Biochemistry 34, 5351-5363
Piller, S. C. et al. (2003) Curr. Drug Targets 4, 409-29
Pommier, Y. et al. (2005) Nat. Rev. Drug Discov. 4, 236-48
Popov, S. et al. (1998) EMBO J. 17, 909-17
Restle, T. et al. (1990) J. Biol. Chem. 265, 8986-8988
Rittinger, K. et al. (1995) Proc. Natl. Acad. Sci. USA 92, 8046-8049
Rodgers, D. W. et al. (1995) Proc. Natl. Acad. Sci. USA 92, 1222-1226
Roisin, A. et al. (2004) J. Biol. Chem. 279, 9208-9214
Ruben, S. et al. (1989) J. Virol. 63, 1-8
Sato, M. et al. (2006). J. Med. Chem. 49, 1506-1508
Semenova, E. A. et al. (2008) Adv. Pharmacol. 56, 199-228
Shimura, K., E. et al. (2008) J. Virol. 82, 764-774
Simon, V. et al. (2006) Lancet 368, 489-504
Sluis-Cremer, N., and Tachedjian, G. (2002) Eur. J. Biochem. 269, 5103-5111
Sluis-Cremer, N. et al. (2002) Mol. Pharmacol. 62, 398-405
Smolov, M. et al. (2006) FEBS J. 273, 1137-51
Sticht, J. et al. (2005) Nat. Struct. Mol. Biol. 12, 671-677
Tachedjian, G., and Goff, S. P. (2003) Curr. Opin. Investig. Drugs 4, 966-973
Tachedjian, G. et al. (2001) Proc. Natl. Acad. Sci. USA 98, 7188-7193
Tachedjian, G. et al. (2005) FEBS Lett. 579, 379-384
Van Maele, B. and Debyser, Z. (2005) AIDS Rev. 7, 26-43.
Venezia, C. F. et al. (2006) Biochemistry 45, 2779-2789
Wain-Hobson, S. et al. (1985) Cell 40, 9-17
Wang, J. et al. (1994) Proc. Natl. Acad. Sci. USA 91, 7242-7246
Wohrl, B. M. et al. (1997) J. Biol. Chem. 272, 17581-17587
Wohrl, B. M. et al. (1999) J. Mol. Biol. 292, 333-344

1. An isolated polypeptide, characterized in that it is selected from the group consisting of:
(a) peptides consisting of or comprising the amino acid sequence X₁X₂ KWX₃TEX₄X₅PLX₆X₇X₈X₉X₁₀ (SEQ ID NO: 17),
wherein:
X₁ is nothing or G,
X₂ is nothing if X₁ is nothing, and X₂ is T if X₁ is G,
X₃ is L or A
X₄ is W or V,
X₅ is I or A if X₄ is W, and X₅ is W if X₄ is V,
X₆ is nothing or T,
X₇ is nothing if X₆ is nothing, and X₇ is nothing or A if X₆ is T,
X₈ is nothing if X₇ is nothing, and X₈ is E if X₇ is A,
X₉ is A if X₆ is T, and X₉ is nothing if X₆ is nothing, and
X₁₀ is E if X₆ is T, and X₁₀ is nothing if X₆ is nothing, and
(b) peptides consisting of the amino acid sequence SEQ ID NO: 1, or consisting of or comprising an amino acid sequence derived therefrom by the substitution of the amino acid at position 1 of SEQ ID NO: 1 by an alanine (A), or the substitution of one of the amino acids at positions 2, 3, 5, 6 and 8-14 of SEQ ID NO: 1 by an alanine (A) or a glycine (G), or the substitution of the amino acid at position 4 of SEQ ID NO: 1 by a glycine (G) or a valine (V),
wherein said isolated polypeptide inhibits in vitro the HIV-1 Reverse Transcriptase polymerase more efficiently than the peptide Pep-A of amino acid sequence SEQ ID NO: 28.

2. A polypeptide according to claim 1, characterized in that it consists of or comprises the amino acid sequence X₁X₂ KWLTEX₃X₄PLX₅X₆X₇X₈X₉ (SEQ ID NO: 34),
wherein:
X₁ is nothing or G,
X₂ is nothing if X₁ is nothing, and X₂ is T if X₁ is G,
X₃ is W or V,
X₄ is I if X₃ is W, and X₄ is W if X₃ is V,
X₅ is nothing or T,
X₆ is nothing if X₅ is nothing, and X₆ is nothing or A if X₅ is T,
X₇ is nothing if X₆ is nothing, and X₇ is E if X₆ is A,
X₈ is A if X₅ is T, and X₈ is nothing if X₅ is nothing, and
X₉ is E if X₅ is T, and X₉ is nothing if X₅ is nothing.

3. A polypeptide according to claim 1, characterized in that the amino acid sequence corresponding to SEQ ID NO: 17 is the amino acid sequence SEQ ID NO: 26.

4. A polypeptide according to claim 1, characterized in that the amino acid sequence corresponding to SEQ ID NO: 17 or SEQ ID NO: 34 is selected from the group consisting of the amino acid sequences SEQ ID NO: 18, 19, 23 to 25 and 27.

5. A polypeptide according to claim 1, characterized in that it further inhibits in vitro the HIV-1 integrase 3′ processing activity.

6. A polypeptide according to claim 5, characterized in that it is selected from the group consisting of the amino acid sequences SEQ ID NO: 18, 19 and 24.

7. A polypeptide according to claim 1, characterized in that the amino acid sequence derived from SEQ ID NO: 1 is selected from the group consisting of SEQ ID NO: 2 to SEQ ID NO: 7, and SEQ ID NO: 9 to SEQ ID NO: 15.

8. A polypeptide according to claim 1, characterized in that it further contains a cysteine residue at the N- or C-terminus.

9. A polypeptide consisting of the amino acid sequences SEQ ID NO: 1 or an amino acid sequence derived therefrom, SEQ ID NO: 17 or SEQ ID NO: 34, as defined in claim 1, characterized in that:
said polypeptide further contains a cysteine residue at the N-terminus or in the case where said polypeptide corresponds to SEQ ID NO: 17 or SEQ ID NO: 34 wherein X₁ and X₂ are respectively G and T, then the amino acid residue at position 2 of said polypeptide is substituted by a cysteine residue, and
said polypeptide further contains a cysteine residue at the C-terminus.

10. A polypeptide according to claim 1, characterized in that one to three amino acid residues thereof is in D conformation.

11. A polypeptide according to claim 1, characterized in that the N- or C-terminal amino acid residue thereof is a in beta conformation.

12. A polypeptide according to claim 1, characterized in that it is coupled to a cell delivery agent.

13. A polypeptide according to claim 12, characterized in that the cell delivery agent is a peptide vector, preferably the peptides having the amino acid sequence SEQ ID NO: 29 (Pep-1), SEQ ID NO: 33 (Pep-3) or SEQ ID NO: 41 (CADY-2c).

14. (canceled)

15. (canceled)

16. (canceled)

17. A composition comprising at least one polypeptide as defined in claim 1 and at least one pharmaceutically acceptable carrier.

18. An in vitro or ex vivo method of inhibiting the reverse transcriptase polymerase activity of HIV-1 reverse transcriptase comprising contacting the reverse transcriptase with at least one polypeptide as defined in claim 1 either in vitro or ex vivo.

19. The method according to claim 18 further comprising inhibiting, in vitro or ex vivo, the HIV-1 integrase 3′ processing activity.

20. The method according to claim 19, characterized in that the polypeptide consists of or comprises the amino acid sequence selected from the group consisting of SEQ ID NO: 18, 19 and 24.

21. An isolated polynucleotide encoding at least one polypeptide as defined in claim 1.

22. A recombinant expression cassette, characterized in that it comprises a polynucleotide as defined in claim 21.

23. A recombinant vector, characterized in that it contains a recombinant expression cassette as defined in claim 22.

24. A host cell, characterized in that it contains a the recombinant vector of claim 23.
